(19) United States
(12) Patent Application Publication
(10) Pub. No.: US 2002/0156039 A1
(43) Pub. Date: Oct. 24, 2002

(54) Diagnosing and treating cancer cells using Sal2

(76) Inventors: Thomas L. Benjamin, Cambridge, MA (US); Dawei Li, Boston, MA (US); Samuel C. Mok, Brookline, MA (US); Daniel W. Cramer, Chestnut Hill, MA (US); Yupo Ma, Sharon, MA (US)

Correspondence Address: CLARK & ELBING LLP, 101 FEDERAL STREET, BOSTON, MA, 02110, US

(21) Appl. No.: 09/988,117

(22) Filed: Nov. 16, 2001

Related U.S. Application Data
(63) Continuation-in-part of application No. 09/812,633, filed on Mar. 19, 2001.
(60) Provisional application No. 60/216,723, filed on Jul. 7, 2000.

(51) Int. Cl. A01K 67/027; A61K 48/00

(52) U.S. Cl. 514/44; 800/10

(57) ABSTRACT

The invention features the use of Sal2 nucleic acids and proteins in methods for treating patients having proliferative disorders, such as cancers, involving mutations in a Sal2 nucleic acid sequence and in the protein that it encodes. In addition, these treatment methods may also be used for patients having a mutation in a nucleic acid sequence encoding a protein that interacts with Sal2 or that functions in a signaling pathway involving Sal2. Furthermore, Sal2 may be used as an anti-viral agent that interferes with the ability of a DNA tumor virus to replicate and disseminate in a cell.

### CROSS REFERENCE TO RELATED APPLICATIONS

This application is a continuation-in-part of U.S. patent application Ser. No. 09/812,633, filed on Mar. 19, 2001, and claims priority from U.S. Provisional Application No. 60/216,723, filed on Jul. 7, 2000, the disclosures of which are hereby included by reference.

### STATEMENT AS TO FEDERALLY SPONSORED RESEARCH

The present research was supported by a grant from the National Cancer Institute (Number R35 CA44343).

### FIELD OF THE INVENTION

The field of the invention is regulation of cellular proliferation.

### BACKGROUND OF THE INVENTION

Transforming genes of DNA tumor viruses perform essential functions in virus growth, acting largely as proto-oncogene activators or tumor suppressor gene inactivators. The isolation and characterization of mutant viruses that are able to propagate in cells containing a mutation in known proto-oncogene or tumor suppressor genes has been useful in identifying and studying the viral equivalents or interactors of these genes. The transforming gene of the highly oncogenic murine polyoma virus was identified through studies of host range mutants isolated using polyoma transformed 3T3 cells as the permissive host and normal 3T3 cells as the non-permissive host. This approach requires expression a known viral protein by the permissive host, since it is based on the idea of complementation between cell-associated wild-type viral genes and an infecting virus mutant. In addition to its use with polyoma virus, the complementation approach has also been successfully used with other oncogenic DNA viruses, e.g., with 293 cells expressing adenovirus E1A/E1B genes and COS cells expressing the SV40 large T antigen. Complementing cell lines have also been used in other systems to propagate specifically defective virus mutants for vaccine development and other purposes. However, by design, these types of systems rely on permissive hosts constructed with known gain-of-function mutations and are only applicable to mutants in known viral genes, as well as to viruses with known mutations, since the host cell must express a functional version of the mutant viral protein.

The use of mutant adenoviruses unable to inactivate p53 or the retinoblastoma protein (pRb) to kill cancer cells lacking one of these proteins has been previously described (Patent Nos. U.S. Pat. No. 5,677,178 and WO 94/18992). It was well known prior to these observations that these two genes are mutated in a variety of cancers.

Unlike the systems described above, we developed a general, unbiased method for identifying new genes involved in the pre-disposition for, or progression of, cancer or other proliferative disorders using tumor host range (T-HR) mutant viruses. While a number of genes are known to be involved in the progression towards cancer, there is a significant need for the identification and characterization of new genes involved in the pre-disposition for, or progression of, cancer or other proliferative disorders. Furthermore, methods for diagnosing and treating patients with mutations in such genes would greatly aid in the management of cancer.

### SUMMARY OF THE INVENTION

The invention features the use of Sal2 nucleic acids and proteins in methods for treating patients having proliferative disorders, such as cancers, involving mutations in a Sal2 nucleic acid sequence and in the protein that it encodes. In addition, these treatment methods may also be used for patients having a mutation in a nucleic acid sequence encoding a protein that interacts with Sal2 or that functions in a signaling pathway involving Sal2. Furthermore, Sal2 may be used as an anti-viral agent that interferes with the ability of a DNA tumor virus to replicate and disseminate in a cell.

Accordingly, the first aspect of the invention features a method of decreasing proliferation of an abnormally proliferating cell, e.g., an ovarian cell. This method includes the step of contacting the abnormally proliferating cell with a Sal2 nucleic acid sequence, where this contacting results in the expression of a Sal2 polypeptide having tumor suppressive activity in the abnormally proliferating cell. In one embodiment, the Sal2 polypeptide includes the amino acid sequence of SEQ ID NO:1 or 3. In another preferred embodiment, the abnormally proliferating cell has a proliferative disease-associated alteration in a Sal2 nucleic acid sequence, for example, one that results in a polypeptide that contains a substitution of a Cys for the Ser at position 73 of SEQ ID NO:1.

The second aspect of the invention features a method of decreasing DNA tumor virus replication and dissemination. This method includes the step of contacting a cell infected with a DNA tumor virus, for example, a simian virus 40, human polyoma virus, herpes virus, primate adenoviruses, parvovirus, or a papilloma virus, with a Sal2 nucleic acid sequence, where this contacting results in the expression of a Sal2 polypeptide in the cell infected with the DNA tumor virus and prevents the DNA tumor virus from replicating and disseminating. In a preferred embodiment of this aspect, the Sal2 polypeptide includes the amino acid sequence provided in SEQ ID NO:1 or 3.

In the third aspect, the invention features an isolated Sal2 nucleic acid sequence, for example, a human Sal2 nucleic acid sequence, encoding a polypeptide that contains a substitution of a Cys for the Ser at position 73 of SEQ ID NO:1.

Definitions

“Tumor host range mutant virus (T-HR mutant),” as used herein, refers to a virus that has a reduced ability to replicate and disseminate in a normal cell, relative to the replication of a wild-type virus in the same type of cell, but is able to replicate and disseminate in a cell having abnormal proliferation. The abnormally proliferating cell may, for example, have one or more mutations in a gene or genes involved in the regulation of cell growth, of the cell cycle, or of programmed cell death (e.g., apoptosis). These genes include, for example, tumor suppressor genes and proto-oncogenes, but any cellular gene that a virus must inactive or activate in order to grow is also included. Adenoviruses having mutations affecting interactions with the p53 and retinoblastoma genes are specifically excluded. In one desirable embodiment, the virus has a mammalian (e.g., rodent or a primate) host range. In a more desirable embodiment the virus has a human host range. A T-HR mutant virus may be, for example, a mutant simian virus 40, human polyoma virus, parvovirus, papilloma virus, herpes virus, or a primate adenovirus. However, any virus that needs to overcome a cell cycle checkpoint or affect signal transduction in order to propagate may be a T-HR mutant virus.

By “DNA tumor virus,” as used herein is meant a virus that has a mammalian (e.g., rodent, primate, or human) host range that can, as a consequence of infecting the cell, transform a normal cell into an abnormally proliferating cell. Examples of “DNA tumor viruses” include simian virus 40, human polyoma virus, parvovirus, papilloma virus, herpes virus, and primate adenovirus.

“T-HR mutant specific for a Sal2 mutation,” as used herein, refers to a TH-R mutant virus that is able to replicate and disseminate in a cell containing a genetic alteration in a Sal2 gene. For example, the “T-HR mutant specific for a Sal2 mutation” may be the TMD-25 T-HR mutant virus described herein. However, a “T-HR mutant specific for a Sal2” mutation may also be unable to replicate and disseminate in a cell containing a genetic alteration in a gene encoding a protein that interacts with Sal2, or in a signaling pathway that involves Sal2.

“Cancer susceptibility gene,” as used herein, refers to any gene that, when altered, increases the likelihood that the organism carrying the gene will develop a proliferative disorder during its lifetime. Examples of such genes include proto-oncogenes, tumor suppressor genes, and genes involved in the regulation of cell growth, the cell cycle, and apoptosis.

By a “Sal2 nucleic acid sequence” as used herein is meant a nucleic acid sequence that is at least 40%, 50%, 60%, 70%, 75%, 80%, 85%, 90%, 95%, or 99% identical to a nucleic acid sequence provided in SEQ ID NO:2 or 4 over a region comprising at least 200, 300, 500, 750, 1000, 1500, 2000, 2500, 3000, or 3500 contiguous nucleotides. In addition a “Sal2 nucleic acid sequence” may be identical to a nucleic acid sequence provided in SEQ ID NO:2 or 4. Desirably, a “Sal2 nucleic acid sequence” is a human or a mouse Sal2 nucleic acid sequence.

By a “Sal2 protein” or a “Sal2 polypeptide,” as used herein is meant an amino acid sequence that is at least 30%, 40%, 50%, 60%, 70%, 75%, 80%, 85%, 90%, 95%, or 99% identical to the amino acid sequence of SEQ ID NO:1 or 3 over a region comprising at least 50, 75, 100, 200, 300, 500, 700, 900, or 1000 contiguous amino acids. In addition a “Sal2 polypeptide” or “Sal2 protein” may be identical to the amino acid sequence provided in SEQ ID NO: 1 or 3. Desirably, a “Sal2 polypeptide” or “Sal2 protein” is a human or a mouse Sal2 polypeptide or amino acid sequence.

By a “Sal2 polypeptide having tumor suppressive activity” is meant a polypeptide that reduces the ability of a TMD-25 T-HR mutant to replicate and disseminate in a cell relative to a Sal2 polypeptide having a substitution of a Cys for the Ser at position 73 of SEQ ID NO:1. In one embodiment, the reduction in the ability of a TMD-25 T-HR to replicate and disseminate is at least 25%. In more desirable embodiments, the reduction in the ability of a TMD-25 T-HR mutant to replicate and disseminate is at least 50%, 75%, 80%, 90%, or 95%. However, a “Sal2 polypeptide having tumor suppressive activity” may also completely block the ability of a TMD-25 T-HR mutant to replicate and disseminate. The reduction in the ability of a TMD-25 T-HR mutant to replicate and disseminate may be measured by determining the number of cells infected with the TMD-25 T-HR mutant that survive in presence of a “Sal2 polypeptide having tumor suppressive activity” in comparison to the number of cells infected with the TMD-25 T-HR mutant that survive in the absence of a “Sal2 polypeptide having tumor suppressive activity,” where an increase in the number of cells surviving is indicative of a reduction in ability of the TMD-25 T-HR mutant to replicate and disseminate.

“Proliferative disease,” as used herein, refers to any disorder that results in the abnormal proliferation of a cell. Specific examples of proliferative diseases are the various types of cancer, such as ovarian cancer. However, proliferative diseases may also be the result of the cell becoming infected with a transforming virus.

“Proliferative disease-associated alteration,” as used herein, refers to any genetic change within a differentiated cell that results in the abnormal proliferation of a cell. In one desirable embodiment, such a genetic change correlates with a statistically significant (p is less than or equal to 0.05) increase in the risk of acquiring a proliferative disease. Examples of such genetic changes include mutations in genes involved in the regulation of the cell cycle, of growth control, or of apoptosis and can further include mutations in tumor suppressor genes and proto-oncogenes. A further example of a proliferative disease-associated alteration may be an alteration in the hSal2 nucleic acid sequence that results in an amino acid substitution in the protein encoded by the nucleic acid sequence, e.g., a substitution of a Cys for the Ser at position 73 of SEQ ID NO:1. However, a “proliferative disease-associated alteration” may also be in a nucleic acid sequence encoding a protein that a p150^{sal2} protein interacts with, or in a nucleic acid sequence encoding a component of a signaling pathway involving p150^{sal2}.

“Abnormal proliferation,” as used herein, refers to a cell undergoing cell division that normally does not undergo cell division.

“Decreasing proliferation,” as used herein, refers to a reduction in proliferation of at least 25%, 50%, 70%, 80%, or 90% when compared with control cells. In a desirable embodiment the reduction in proliferation is at least 95%. In a more desirable embodiment the reduction in proliferation is 100%.

The term “alteration,” when used herein, in reference to a gene, refers to a change in the nucleic acid sequence. Such a change may include, for example, insertions, deletions, and substitutions of one or more nucleic acids, as well as inversions and duplications.

“Genetic lesion,” as used herein, refers to a nucleic acid change. Examples of such a change include single nucleic acid changes as well as deletions and insertions of one or more nucleic acid. However, genetic lesions can also include duplications and inversions. In addition, a genetic lesion may be a naturally-occurring polymorphism, for example, one that predisposes an organism carrying the polymorphism to acquiring a proliferative disease.

“Polymorphism,” as used herein, refers to an alteration in a nucleic acid sequence, for example, a gene. Such an alteration may result in a codon change, which in turn may result in, for example, the substitution of a Cys for the Ser at position 73 of SEQ ID NO:1.

“Modification of function,” as used herein, refers to a change in the function of the protein. Such a change can, for example, result in the partial or complete loss of function, but it can also result in a gain of function.

“Sal2 mutation”, as used herein, refers to a genetic change in the nucleic acid sequence of a Sal2 gene, for example, SEQ ID NO:2 and SEQ ID NO:4, which results in the abnormal proliferation, or predisposition to abnormal proliferation, of a cell carrying such a change. In a desired embodiment, the genetic change is a missense mutation or a mutation which lowers the tumor suppressor activity of the encoded protein. In a more desired embodiment, the mutation is a substitution of a Cys for the Ser at position 73 of SEQ ID NO:1.

As used herein, the term “promoter” is intended to encompass transcriptional regulatory elements, that is, all of the elements that promote or regulate transcription, including core elements required for basic interactions between RNA polymerase, transcription factors, upstream elements, enhancers, and response elements.

“Operably linked,” as referred to herein, describes the functional relationship between nucleic acid sequences, for example, a promoter sequence, and a gene to be expressed. Operably linked nucleic acids may be part of a contiguous sequence. However a physical link is not necessary for two nucleic acid sequences to be operably linked. For example, enhancers can exert their effect over long distances and therefore do not require a physical link in sequence to the gene whose transcription they affect.

Reference herein to the “transcriptional regulatory elements” of a gene or a class of genes includes both the entire gene as well as an intact region of naturally-occurring transcriptional regulatory elements. Also included are transcription regulatory elements modified by, for example, rearrangement of the elements, deletion of some elements or of extraneous sequences, and insertion of heterologous elements.

The term “knockout,” as used herein, refers to an alteration in the sequence of a specific gene that results in a decrease of function of that gene. In one desirable embodiment, the alteration results in undetectable or insignificant expression of the gene and in a complete or partial loss of function. Furthermore, the disruption may be conditional, e.g., dependent on the presence of tetracycline. Knockout animals may be homozygous or heterozygous for the gene of interest. In addition, the term knockout includes conditional knockouts, where the alteration of the target gene can occur, for example, as a result of exposure of the animal to a substance that promotes target gene alteration, introduction of an enzyme that promotes recombination at the target gene site (e.g., Cre in the Cre-lox system, or FLP in the FLP/FRT system), or any other method for directing target gene alteration.

“Conditionally expressed,” as used herein, refers to any method that may be used to control expression of a gene, such as a transgene. These methods may, for example, include the use of promoters that are regulated a substance, such as tetracycline, that can be administered to the organism, or of promoters that are only active at certain stages of development or in certain tissues. In addition, conditional expression may involve inactivating a gene, for example, by FLP/FRT- or Cre-lox-mediated recombination.

The term “restriction fragment length polymorphism (RFLP) analysis,” as used herein, refers to a method of determining whether an organism carries a specific nucleic acid sequence, for example, a specific alteration in a gene. This method may involve, for example, amplification of a nucleic acid from the organism, followed by cleavage of the nucleic acid with an enzyme, such as a restriction enzyme, and visualizing the products of the cleavage reaction. Furthermore, the cleavage products may be compared to control reactions.

As used herein, “alters proliferation” refers to any change in the proliferation of a cell. For example, this term can be used to describe an increase or a decrease in the rate of cell division. In addition, an alteration of proliferation may refer to a normally quiescent cell entering into the cell cycle or a normally dividing cell ceasing to enter into the cell cycle.

By “reduced ability to replicate and disseminate,” as used herein is meant a reduction in the ability of a virus to replicate and disseminate, relative to a wild-type virus in the same type of cell, of at least 50%, 60%, 70%, 80%, 90%, 95%, 99% or the complete inability to replicate or disseminate. Desirably, the ability to replicate and disseminate is reduced by at least 90%, 95%, or 99%.

By “decreasing replication and dissemination,” as used herein is meant a reduction in virus replication and dissemination in the presence of a compound, for example, a Sal2 nucleic acid having tumor suppressive activity, that is at least 50%, 60%, 70%, 80%, 90%, 95%, 99% less than the amount of virus replication and dissemination seen under control conditions in the absence of the compound. Desirably, replication and dissemination is reduced by at least 90%, 95%, or 99%.

“Measuring protein levels,” as used herein, includes any standard assay used in the art to either directly or indirectly determine protein levels. Such assays, for example may include the use of an antibody, Western analysis, Bradford assays, and spectrophotometric assays.

“Measuring nucleic acid levels,” as used herein, includes any standard assay used in the art to either directly or indirectly determine nucleic acid levels. Such assays include, for example, hybridization analysis, gel electrophoresis, Northern blots, Southern blots, and spectrophotometric assays.

By a “substantially pure polypeptide” is meant a polypeptide (for example, a Sal2 polypeptide) that has been separated from components that naturally accompany it. Typically, the polypeptide is substantially pure when it is at least 60%, by weight, free from the proteins and naturally-occurring organic molecules with which it is naturally associated. In one desirable embodiment, the preparation is at least 75%. In more desirable embodiments, the preparation is at least 90% or at least 99%, by weight, a Sal2 polypeptide. A substantially pure Sal2 polypeptide may be obtained, for example, by extraction from a natural source (for example, a mammalian cell); by expression of a recombinant nucleic acid encoding a Sal2 polypeptide; or by chemically synthesizing the protein. Purity can be measured by any appropriate method, for example, column chromatography, polyacrylamide gel electrophoresis, or by HPLC analysis.

By an “isolated nucleic acid sequence” is meant a nucleic acid sequence that is free of the nucleic acid sequence which, in the naturally-occurring genome of the organism from which the nucleic acid sequence of the invention is derived, flank the nucleic acid sequence. The term therefore includes, for example, an mRNA; a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. It also includes a recombinant DNA that is part of a hybrid gene encoding additional polypeptide sequence.

By a “candidate compound” or “test compound” is meant a chemical, be it naturally-occurring or artificially-derived, that is surveyed for its ability to modulate cell proliferation, by employing one of the assay methods described herein. Candidate compounds may include, for example, peptides, polypeptides, synthetic organic molecules, naturally-occurring organic molecules, nucleic acid molecules, and components thereof.

By “high stringency hybridization conditions” is meant, for example, hybridization at approximately 42° C. in about 50% formamide, 0.1 mg/ml sheared salmon sperm DNA, 1% SDS, 2×SSC, 10% Dextran sulfate, a first wash at approximately 65° C. in about 2×SSC, 1% SDS, followed by a second wash at approximately 65° C. in about 0.1×SSC. Alternatively, “high stringency hybridization conditions” may include hybridization at approximately 42° C. in about 50% formamide, 0.1 mg/ml sheared salmon sperm DNA, 0.5% SDS, 5×SSPE, 1×Denhardt's, followed by two washes at room temperature in 2×SSC, 0.1% SDS, and two washes at between 55-60° C. in 0.2×SSC, 0.1% SDS.

Advantages

The use of a T-HR mutant virus has a particular advantage over standard chemotherapy treatments, and the like, in that it is specific for cells with a proliferative disease. Therefore, one would expect this type of therapy to have fewer toxic side effects than the chemotherapeutic agents used today.

### BRIEF DESCRIPTION OF THE DRAWINGS

FIG. 1 is a series of photographs depicting the growth of wild-type polyoma virus and the TMD25 virus on host cells.

FIG. 2A is sequence comparison between a region of the wild-type and TMD25 polyoma virus large T antigen nucleic acid and amino acid sequences and shows the 20 bp sequence duplication responsible for the TMD25 mutation.

FIG. 2B is a schematic diagram of various mSal2 clones obtained in a yeast two-hybrid assay, as well as a series of images showing the interaction of a mSal2 clone with wild-type polyoma virus large T antigen protein and the lack of interaction with TMD25 virus large T antigen protein in a yeast two-hybrid assay.

FIG. 2C is a series of large T antigen amino acid sequences and shows the deletion analysis of the TMD25 mutant.

FIG. 3A is a schematic diagram showing the regions of the mSal2 gene used to develop antibodies.

FIG. 3B is a series of Western blots using protein from mouse and human cells that show the mSal2 gene product to be p150^{sal2}.

FIG. 3C is a series of Western blots of extracts from human 293 and U2OS cells that was first probed with an antiserum against the mSal2 carboxyl-terminus. The filter was then stripped and re-probed with an antibody against the mSal2 amino-terminus.

FIG. 4A is a picture of a protein gel showing that mSal2 binds to wild-type polyoma virus, but not to TMD25 large T protein in vitro.

FIG. 4B is a series of protein gels showing the interaction of mSal2 and wild-type, but not of mSal2 and TMD-25 mutant, large T protein in transfected 3T3 cells. These results are confirmed in BMK cells infected with wild-type polyoma virus and with TMD25 mutant virus.

FIG. 5A is a series of images showing the failure of TMD25 to replicate in newborn mice.

FIG. 5B is a series of Southern blots showing that TMD25 fails to replicate in BMK cells and that p150^{sal2} represses viral origin replication.

FIG. 5C is a Southern blot showing that inhibition of replication by mSal2 requires the large T interaction domain and can be overcome by wild-type, but not mutant large T antigen.

FIG. 6 is a Western blot showing mSal2 expression in various mouse tissues.

FIG. 7 is a Western blot showing hSal2 expression in human ovarian tumors.

FIG. 8 is a Western blot showing expression of p150^{sal2} in human 293 cells.

FIG. 9 is a series of images showing immunostaining of p150^{sal2} in human ovary tissue (A) and in ovarian tumors (B).

FIG. 10A is a series of images and a bar graph showing that p150^{sal2} suppresses growth of human ovarian tumor cells, which is indicated by a reduction in BrdU incorporation in p150^{sal2} transfected cells.

FIG. 10B is a series of images showing a colony reduction assay that indicates that cells transfected with p150^{sal2} are less viable than control transfected cells.

FIG. 11 is an agarose gel showing that the 73S allele is lost in some ovarian tumors.

### DETAILED DESCRIPTION OF THE INVENTION

The invention features the use of Sal2 nucleic acids and proteins in methods for treating patients having proliferative disorders, such as cancers, involving mutations in a Sal2 nucleic acid sequence and in the protein that it encodes. In addition, these treatment methods may also be used for patients having a mutation in a nucleic acid sequence encoding a protein that interacts with Sal2 or that function in a signaling pathway involving Sal2. Furthermore, Sal2 may be used as an anti-viral agent that interferes with the ability of a DNA tumor virus to replicate and disseminate in a cell.

### Identifying Genes Altered in Cancerous Cells

Host Range Selection of Viruses

The present invention describes the use of tumor host range mutant viruses (T-HR mutants) that are capable of replicating in abnormally proliferating cells but not in normal cells. Therefore, these viruses are useful for identifying genes altered in abnormally proliferating cells. T-HR mutants generally have a mutation that causes a modification of function of the protein encoded by that gene. These mutations typically lie in the transforming genes of the DNA tumor viruses and are usually activators of cellular proto-oncogenes or inactivators of tumor suppressor genes. T-HR mutants may be isolated based on their ability to propagate (i.e., to replicate and disseminate) only in tumor cells that have mutations in the cellular protein that is normally targeted by the viral transforming protein. However, while permissive hosts for T-HR mutants could fail to express, or carry a mutation in, the cellular target itself, these hosts, or other cancer cells, may alternatively be defective in some interacting partner or effector of the target gene. Examples of the latter possibility are, for example, permissive hosts for polyoma or adenovirus mutants that are defective in binding pRb or p53, despite expressing these tumor suppressors themselves, as a result of defects in INK4A gene products which impinge on pRb and p53 (Freund et al., J. Virol. 68:7227-7234, 1994; Harvey and Levine, Genes & Dev. 5:2375-2385, 1991; Yang et al., Cancer Res. 61:5959-5963, 2001; McCormick, Oncogene 19:6670-6672, 2000; Linardopoulos et al., Cancer Res. 55:5168-5172, 1995).

The methods of the invention have been applied to a ‘tumor host range’ selection procedure using the polyoma virus as a tool to search for new interactions of viral proteins, e.g., T antigens, with cellular proteins. The rationale behind this approach is based on the idea that genetic changes in tumor cells resulting in a modification of function of the cellular protein can provide the basis for a search to uncover new viral functions and interactions with cellular targets. In principle, ‘Tumor host range’ selection could reveal mutations in other functions, e.g., VP1, 2 or 3 involving interactions with receptors or the cellular machinery involved in virus uptake, uncoating or transport to the nucleus, or even in some aspect of virus assembly, or enhancer mutations that lead to alterations in enhancer function.

For example, alterations in yet unknown targets of viral genes might occur in spontaneous tumors or non-virally transformed cells. This suggests a rationale for isolating T-HR mutants based on modification of function in cancer cells. Mutants selected to grow in tumor cells, but not in normal cells, are useful for identifying new viral gene functions and their cellular targets. Targets identified in this way may include products of tumor suppressor genes or proto-oncogenes or any factor expressed in normal cells, which the virus must inactivate in order to propagate, but that is no longer expressed in tumor cells.

Identification of mSal2

The utility of the T-HR mutant based approach for identifying new genes involved in the susceptibility to proliferative diseases is shown by the identification of mSal2. The use of a T-HR mutant coupled with the power of the yeast two-hybrid screen resulted in the identification of a cellular target protein. Using T-HR mutants to identify cell cycle regulatory proteins is advantageous on two levels; first, in choosing an appropriate wild-type ‘bait’ corresponding to the region altered in the mutant, and second, in enabling a counterscreen where lack of interaction with the mutant is helpful in identifying cellular target(s) relevant to the mutant phenotype and possibly also to the transformed state of the permissive host. One embodiment of the general protocol included as an aspect of the invention is outlined in Table 1 below.

TABLE 1
Tumor Host Range Mutants-Selection Procedure and Target Identification

I. Mutant Selection

| | |
|---|---|
| 1. | Random mutagenesis of wild-type viral DNA |
| 2. | Amplification of the mutant virus by growth in tumor cells |
| 3. | Cloning by plaque isolation on tumor cells |
| 4. | Screening of plaque lysates for the absence of growth in normal cells |
| 5. | Molecular cloning and sequencing of the mutant viral DNA |

II. Target Identification and Validation

| | |
|---|---|
| 6. | Screening of a mouse embryo cDNA library in yeast with wild-type |
| | bait |
| 7. | Counterscreening positive clones for lack of interaction with mutant |
| | bait |
| 8. | Construction of complete cDNA expressing the target protein |
| 9. | Verification of viral protein-cellular target interactions in vitro and in |
| | vivo (e.g., T antigen-cellular protein interactions). |

III. Identification of Risk Factors

| | |
|---|---|
| 10. | Sequencing DNA derived from a tumor |
| 11. | Sequencing DNA derived from normal tissue of the same patient |
| 12. | Using the sequence information to establish whether the mutation is |
| | somatic of germline |
| 13. | Using this information in an epidemiological study to assess risk |
| | factors in a population |

What follows is an illustration of the use of the methods of the invention to identify a new target of large T antigen, referred to as mSal2, using T-HR mutants of the polyomavirus. First, tumor host range selection identified a host range mutant of the polyomavirus that is able to grow in certain tumor or transformed cells but not in normal cells. The mutant virus encodes an altered large T antigen protein and is defective in replication and tumor induction in newborn mice. Next, mSal2 was identified as a binding target of the polyoma virus large T antigen through a yeast two-hybrid screen. mSal2 shows no interaction with the mutant large T antigen. Specifically, the mutant virus fails to bind mSal2 and is unable to propagate or to induce most of the tumor types in the mouse that the wild-type virus typically induces.

The gene product p150^{sal2} is expressed in a number of mouse and human tissues. It is found in nuclei of germinal epithelial cells from normal human ovary but is missing or altered in ovarian carcinomas derived from these cells (Table 3). Using an antibody to mSal2 that cross-reacts with the human protein, Sal2 was shown to be expressed as a protein of approximately 150 kDa in several normal murine and human tissues. Normal human ovarian epithelial cells show strong nuclear staining with the antibody. A majority of ovarian carcinomas derived from these cells show no detectible p150^{sal2} by Western analysis and are negative by in situ immunochemistry. Some tumors display diffuse cytoplasmic, rather than nuclear, staining. (See Examples below.)

mSal2 is a zinc finger protein and a putative transcription factor that may have a role as a tumor suppressor. mSal2 is homologous to the Drosophila homeotic gene spalt and to sal homologues identified in several vertebrate species (see below). The human homologue of the Drosophila spalt gene, hSal2, has been mapped adjacent to, or overlapping with, a chromosomal region associated with a loss of homozygosity in ovarian and other cancers.

The spalt or sal gene family of transcription factors is conserved in evolution from flies to man. First identified in Drosophila, spalt is a region-specific homeotic gene which functions in specifying anterior and posterior structures in the early embryo (Kuhnlein et al., EMBO J 13:168-179 (1994); Jurgens et al., EMBO J 7:189-196 (1988)) and also in later stages of organogenesis (Kuhnlein et al., Mech. Dev. 66:107-118 (1997); Barrio et al., Dev. Biol. 215:33-47 (1999)). spalt-related sal genes have been identified and studied in worms (Basson et al., Genes Dev. 10:1953-1965 (1996)), fish (Koster et al., Development 124:3147-3156 (1997)), frogs (Hollemann et al., Mech. Dev. 55:19-32 (1996); Onuma, Biochem. Biophys. Res. Commun. 264:151-156 (1999)), mice (Ott et al., Mech. Dev. 56:117-128 (1996); Kohlhase et al., Nat. Genet. 18:81-83 (2000)) and man (Kohlhase et al., Genomics 38:291-298 (1996); Kohlhase et al., Genomics 1:216-222 (1999); Kohlhase et al., Cytogenet. Cell Genet. 84:31-34 (1999)). In humans, a defect in the hSal1 gene underlies the multiple developmental defects seen in Townes-Brocke syndrome (Kohlhase et al., Nat. Genet. 18:81-83 (1998)). Sal proteins contain multiple Zinc fingers, which frequently occur as C2H2 pairs with a conserved motif (Kuhnlein et al., EMBO J 13:168-179 (1994)). mSal2 has a structural arrangement typically seen in vertebrates with a single finger (C3H) near the amino-terminus and a cluster of three fingers (C2H2) considered essential for DNA binding in the middle portion of the protein (Pabo et al., Annu. Rev. Biochem. 61:1053-1095 (1992)). Like other Sal proteins, mSal2 has both glutamine-rich and proline- and alanine-rich sequences consistent with its transcriptional activator and repressor functions.

Although it has been shown in several species that Sal family transcription factors play important roles in embryonic development, downstream target genes have yet to be identified. Nevertheless, two important signaling pathways lying upstream of sal have been recognized. Regulation of spalt occurs in part through dpp, a member of the TGF-β family, which functions as a ‘gradient morphogen’ in the early Drosophila embryo (de Celis et al., Nature 381:421-424 (1996); Lecuit et al., Nature 381:387-393 (1996); Nellen et al., Cell 85:357-368 (1996)). In Medaka, Sal1 expression occurs in response to hh (hedgehog) and is downregulated through PK-A (Koster et al., Development 124:3147-3156 (1997)). The TGF-β family of polypeptides has well known inhibitory effects on epithelial cell growth and survival. Disruptions in signaling pathways initiated by TGF-β are known to occur in some cancers (Kretzschmar et al., Current Opinion in Genetics & Development 8:103-111 (1998); Serra et al., Nature Med. 2:390-391 (1996)). In particular, mutations in SMAD genes, essential mediators of signaling via TGF-β receptors, have been linked to pancreatic, colorectal, and other cancers (Eppert et al., Cell 86:543-552 (1996); Hahn et al., Science 271:350-353 (1996); Schutte et al., Cancer Res. 56:2527-2530 (1996)). Similarly, disruptions in signaling via ‘hedgehog’ ligands and their ‘patched’ receptors are important in development of basal cell carcinoma (Hahn et al., Cell 85:841-851 (1996); Johnson et al., Science 272:1668-1671 (1996); Oro et al., Science 276:817-821 (1997); Stone et al., Nature 384:129-134 (1996)).

### Diagnosis

Diagnosis and Risk Assessment

In addition to helping identify genes that are altered in cancerous cells, target gene profiles can also be used to diagnose and/or stage various proliferative disorders and for diagnosing pre-symptomatic genetic lesions in normal tissues. The methods of the present invention can be used to diagnose cancerous cells in a patient by determining whether the cells of the patient can act as permissive hosts for the growth of a mutant virus, particularly a T-HR mutant. As described above, a permissive host for the growth of a mutant virus (e.g., a mutant virus that lacks a functioning transforming protein) has a mutation in a cellular gene that is the target for the wild-type viral protein that corresponds to the mutant viral protein. This cellular mutation is believed to compensate for the modification of function in a particular gene in the T-HR mutant and contribute to the cancerous phenotype of the cell.

Once a target protein has been identified, tests for the lack of interaction of the cellular protein with the mutant viral protein are used to confirm the specificity of the interaction of the cellular protein with the wild-type (transforming) protein. A lack of interaction indicates that binding of the wild-type viral protein to the cellular protein is specific. Protein interaction can be verified by numerous methods know to those skilled in the art, including, for example, yeast two-hybrid assays, GST-pull down assays, co-immunoprecipitation, and Far-Western analysis. General guidance regarding these techniques can be found in standard laboratory manuals, such as Ausubel et al. (Current Protocols in Molecular Biology, John Wiley & Sons, New York, N.Y., (1994)), and Sambrook et al. (Molecular Cloning: A Laboratory Manual, Cold Spring Harbor Laboratory, N.Y., (1989)). Once an interaction between the wild-type viral protein and the cellular protein is confirmed, the complete gene and gene product can readily be identified by those skilled in the art using, for example, the methods described below.

The present invention recognizes that the T-HR mutant selection procedures identified herein may identify mutant cellular genes, and their encoded protein products, e.g., cellular genes encoding cell cycle proteins, tumor suppressors, proto-oncogenes, transcriptional factors, regulators of apoptosis, etc., that have genetic lesions associated with a particular proliferative disorder. Those skilled in the art will appreciate that many proliferative disorders, such as cancers, correlate with a particular mutation or mutations in the DNA of a patient. By comparing the sequence for a particular gene in both normal and tumor tissue from the same patient, one can determine if the mutation is of somatic or germline origin. This information that may be used to screen a population as a whole for individuals that are at an increased risk of developing a particular type of proliferative disorder.

The present invention provides a method of identifying a genetic lesion in a cell by determining whether a cell can act as a permissive host for the growth of a particular T-HR mutant, such a T-HR mutant virus being capable of growing on a cell having a specific genetic lesion and not being capable of growth on a cell lacking this genetic lesion. This type of information may even be used to further characterize the cancer cell (e.g., to grade the stage to which the cancer has progressed).

In addition, the cellular gene that encodes a protein that is a target for a viral transforming protein may also be analyzed to determine whether there is a genetic lesion in the cellular gene. Such a genetic lesion may be associated with a particular cancer. As noted above, a genetic lesion in the Sal2 gene has been identified by the present invention that may be associated with ovarian cancer. Specifically, this genetic lesion, resulting in the substitution of a Cys for the Ser at position 73 in protein encoded by the mSal2 gene of SEQ ID NO:4, has been identified in DNA from blood samples from patients with ovarian cancer. Probes and primers based on this genetic lesion may be used as markers to detect the Ser73Cys change in samples from other patients.

A genetic lesion in a candidate gene may be identified in a biological sample obtained from a patient using a variety of methods available to those skilled in the art. Generally, these techniques involve PCR amplification of nucleic acid from the patient sample, followed by identification of the genetic lesion by either altered hybridization, aberrant electrophoretic gel migration, restriction fragment length polymorphism (RFLP) analysis, binding or cleavage mediated by mismatch binding proteins, or direct nucleic acid sequencing. Any of these techniques may be used to facilitate detection of a genetic lesion in a candidate gene, and each is well known in the art; examples of particular techniques are described, without limitation, in Orita et al. (Proc. Natl. Acad. Sci. USA 86:2766-2770, 1989) and Sheffield et al. (Proc. Natl. Acad. Sci. USA 86:232-236 (1989)). Furthermore, expression of the candidate gene in a biological sample (e.g., a biopsy) may be monitored by standard Northern blot analysis or may be aided by PCR (see, e.g., Ausubel et al., Current Protocols in Molecular Biology, John Wiley & Sons, New York, N.Y. (1994); PCR Technology: Principles and Applications for DNA Amplification, H. A. Ehrlich, Ed., Stockton Press, NY; Yap et al., Nucl. Acids. Res. 19:4294 (1991)).

Once a genetic lesion is identified using the methods of the invention (as is described above), the genetic lesion is analyzed for association with an increased risk of developing a proliferative disorder. In this respect, the present invention provides a method of detecting the presence of a genetic lesion in the human Sal2 gene in a physiological sample, however the method is not limited to this one gene, but rather can be applied to any gene that is associated with an increased risk for developing a proliferative disorder.

Furthermore, antibodies against a protein produced by the gene included in the genetic lesion, for example the Sal2 protein. Antibodies may be used to detect altered expression levels of the protein, including a lack of expression, or a change in its mobility on a gel, indicating a change in structure or size. In addition, antibodies may be used for detecting an alteration in the expression pattern or the sub-cellular localization of the protein. Such antibodies include ones that recognize both the wild-type and mutant protein, as well as ones that are specific for either the wild-type or an altered form of the protein, for example, one encoded by a polymorphic Sal2 gene. Monoclonal antibodies may be prepared using the Sal2 proteins described above and standard hybridoma technology (see, e.g., Kohler et al., Nature 256:495 (1975); Kohler et al., Eur. J. Immunol. 6:511 (1976); Kohler et al., Eur. J. Immunol. 6:292 (1976); Hammerling et al., In Monoclonal Antibodies and T Cell Hybridomas, Elsevier, New York, N.Y. (1981); Ausubel et al., Current Protocols in Molecular Biology, John Wiley & Sons, New York, N.Y. (1994)). Once produced, monoclonal antibodies are also tested for specific Sal2 protein recognition by Western blot or immunoprecipitation analysis (by the methods described in, for example, Ausubel et al. (Current Protocols in Molecular Biology, John Wiley & Sons, New York, N.Y. (1994)).

Antibodies used in the methods of the invention may be produced using amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson and Wolf (CABIOS 4:181 (1988)). These fragments can be generated by standard techniques, e.g., by the PCR, and cloned into the pGEX expression vector (Ausubel et al., Current Protocols in Molecular Biology, John Wiley & Sons, New York, N.Y. (1994)). GST fusion proteins are expressed in E. coli and purified using a glutathione agarose affinity matrix as described in Ausubel et al. (Current Protocols in Molecular Biology, John Wiley & Sons, New York, N.Y., (1994)).

To generate rabbit polyclonal antibodies, and to minimize the potential for obtaining antisera that is non-specific, or exhibits low-affinity binding, two or three fusions are generated for each protein, and each fusion is injected into at least two rabbits. Antisera are raised by injections in series, with at least three booster injections being desirable. These methods for antibody production and characterization are applicable to any other protein that is identified by the methods of the invention.

The antibody may be used in immunoassays to detect or monitor protein expression, e.g., Sal2 protein expression, in a biological sample. A polyclonal or monoclonal antibody (produced as described above) may be used in any standard immunoassay format (e.g., ELISA, Western blot, or RIA) to measure polypeptide levels. These levels may be compared to normal levels. Examples of immunoassays are described, e.g., in Ausubel et al. (Current Protocols in Molecular Biology, John Wiley & Sons, New York, N.Y. (1994)). Immunohistochemical techniques may also be utilized for protein detection. For example, a tissue sample may be obtained from a patient, sectioned, and stained for the presence of Sal2 using an anti-Sal2 antibody and any standard detection system (e.g., one which includes a secondary antibody conjugated to horseradish peroxidase). General guidance regarding such techniques can be found in, e.g., Bancroft and Stevens (Theory and Practice of Histological Techniques, Churchill Livingstone (1982); and Ausubel et al., Current Protocols in Molecular Biology, John Wiley & Sons, New York, N.Y. (1994)).

Use of hSal2 as a Diagnostic Tool

As an example of the utility of this approach, the likelihood that hSal2 functions as a tumor suppressor for ovarian cancer has been explored directly by screening a number of ovarian carcinomas for expression of p150^{sal2} and for mutations in the gene. Approximately 80% of the tumors examined were negative or showed altered or reduced patterns of expression by Western analysis. Immunolocalization in frozen tissue sections showed strong staining in nuclei of epithelial cells on the surface of the normal ovary. In most instances, tumor cells showed a complete lack of staining. Cytoplasmic rather than nuclear staining was seen in some areas of otherwise negative tumors. A limited screen for mutations in hSal2 uncovered point mutations in four cases. Cytogenetic approaches and major sequencing effort may be carried out using microsatellite markers. Such approaches have been used to map hSal2 adjacent to, and possibly overlapping with, a chromosomal region associated with loss of homozygosity in ovarian (Bandera et al., Cancer Res. 57:513-515 (1997)) and other cancers, e.g., bladder cancer (Chang et al., Cancer Res. 55:3246-3249 (1995)).

The mSal2 gene identified by the present invention may be used to further elucidate the cellular pathways of tumor suppression that regulate key cell cycle events. Alternatively, mSal2 may be used to screen for potential tumors, e.g., lung tumors, brain tumors, stomach tumors, prostate tumors; ovarian tumors, tumors in SCID mice, as well as in knockout or transgenic animals, as discussed in detail below.

Treatment

In addition to providing a method for identifying genes altered in cancer cells and diagnosing patients who carry such mutation, the invention further provides a method of killing an abnormally proliferating cell using a tumor host range mutant virus.

For example, T-HR mutants can be used to specifically target and kill cancer cells in an organism. Since these viruses can only propagate in cells that carry a mutation in a cellular gene that the virus would normally have to activate, in the case of proto-oncogene, or inactivate, in the case of a tumor suppressor gene, in order to propagate, such a virus would be specific to abnormal cells. Therefore, T-HR mutants can be used to specifically eliminate cancer cells from a patient. For example, a T-HR mutant (i.e., a polyoma virus carrying an altered large T antigen causing it to be defective in replication and tumor induction) may be used to selectively kill human ovarian cancer cells that carry a genetic lesion in the hSal2 gene, such Ser73Cys substitution described above.

However, one skilled in the art would realize that any number of genes, including ones involved in cell growth, cell cycle regulation, and apoptosis, may be altered in cancer cells. The methods of the invention are applicable to any alteration in a cancer cell that allows a T-HR mutant to grow. Therefore, any cancer that enables a T-HR mutant to propagate can be treated according to the methods of the invention disclosed herein.

The therapeutic T-HR mutant may be administered by any of a variety of routes known to those skilled in the art, such as, for example, intraperitoneal, subcutaneous, parenteral, intravenous, intramuscular, or subdermal injection. However, the T-HR mutant may also be administered as an aerosol, as well as orally, nasally, or topically. Standard concentrations used to administer a T-HR mutant include, for example, 10², 10³, 10⁴, 10⁵, or 10⁶ plaque forming units (pfu)/animal, in a pharmacologically acceptable carrier. Appropriate carriers or diluents, as well as what is essential for the preparation of a pharmaceutical composition are described, e.g., in Remington's Pharmaceutical Sciences (18^{th} edition), ed. A. Gennaro, 1990, Mack Publishing Company, Easton, Pa., a standard reference book in this field.

Formulations for parenteral administration may, for example, contain excipients, sterile water, or saline. For inhalation, formulations may contain excipients, for example, lactose. Aqueous solutions may be used for administration in the form of nasal drops, or as a gel for topical administration. The exact dosage used will depend on the severity of the condition (e.g., the size of the tumor), or the general health of the patient and the route of administration. The T-HR mutant may be administered once, or it may be repeatedly administered as part of a regular treatment regimen over a period of time.

In addition, the invention provides methods of halting abnormal proliferation in a cell using a target gene of a tumor virus that normally functions as a tumor suppressor gene. Such target genes, for example, mSal2, may be identified using a T-HR according to the methods of the invention. A mSal2 nucleic acid sequence, or a nucleic acid sequence encoding a protein that interacts with mSal2, may be introduced into an abnormally proliferating cell, for example, by using liposome-based transfection techniques, to treat the proliferative disorder (Units 9.1-9.4, Ausubel et al., Current Protocols in Molecular Biology, John Wiley & Sons, New York (1995)). In addition, such DNA constructs may also be introduced into mammalian cells using an adenovirus, or retroviral or vaccinia viral vectors (Units 9.10 and 16.15-16.19, Ausubel et al., Current Protocols in Molecular Biology, John Wiley & Sons, New York (1995)). These standard methods of introducing DNA into cells are applicable to a variety of cell-types.

For example, recombinant adenoviral vectors offer several significant advantages for gene transfer. The viruses can be prepared at extremely high titer, infect non-replicating cells, and confer high-efficiency and high-level transduction of target cells in vivo after directed injection or perfusion. Either directed injection or perfusion would be appropriate for delivery of vectors containing a T-HR target gene in a clinical setting. Moreover, transient expression may be sufficient to remove the abnormally proliferating cells and it may be desirable in view of possible bio-safety or toxicity concerns associated with long-term expression of a T-HR target gene.

In animal models, adenoviral gene transfer has generally been found to mediate high-level expression for approximately one week. The duration of transgene expression may be prolonged, and ectopic expression reduced, by using tissue-specific promoters. Other improvements in the molecular engineering of the adenoviral vector itself have produced more sustained transgene expression and less inflammation. This is seen with so-called “second generation” vectors harboring specific mutations in additional early adenoviral genes and “gutless” vectors in which virtually all the viral genes are deleted utilizing a Cre-Lox strategy (Engelhardt, et al., Proc. Natl. Acad. Sci. USA 91:6196-6200, 1994; Kochanek, et al., Proc. Natl. Acad. Sci. USA 93:5731-5736, 1996).

In addition, recombinant adeno-associated viruses (rAAV), derived from non-pathogenic parvoviruses, may be used to express a T-HR target gene as these vectors evoke almost no cellular immune response, and produce transgene expression lasting months in most systems. Incorporation of a tissue-specific promoter is, again, beneficial. Furthermore, besides adenovirus vectors and rAAVs, other vectors and techniques are known in the art, for example, those described by Wattanapitayakul and Bauer (Biomed. Pharmacother. 54:487-504, 2000), and citations therein.

A vector carrying a T-HR target gene can be delivered to the target organ through in vivo perfusion by injecting the vector into the target organ, e.g., the ovary, or into blood vessels supplying this organ (e.g., for the liver, the portal vein (Tada, et al., Liver Transpl. Surg. 4:78-88, 1998) could be used.

Furthermore, a target gene of a T-HR mutant, for example, mSal2, may also be used as an anti-viral agent. In the case of a tumor suppressor gene, a DNA tumor virus needs to inactivate the gene to replicate and disseminate. Accordingly, providing active forms of such genes to a cell, or over-expressing these genes in the cell, would effectively interfere with virus replication and dissemination, and, thereby, prevent the virus from causing or contributing to a proliferative disorder. Alternatively, an anti-sense nucleic acid for a proto-oncogene may be used to inactivate such a gene in a cell and, thereby, prevent a DNA tumor virus from activating the gene, or in the case of an abnormally proliferating cell, to halt abnormal proliferation.

### Test Compounds

Compounds that may be tested for an effect on proliferative diseases can be from natural as well as synthetic sources. Those skilled in the field or drug discovery and development will understand that the precise source of test extracts or compounds is not critical to the methods of the invention. Examples of such extracts or compounds include, but are not limited to, plant-, fungal-, prokaryotic-, or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceanographics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

Transgenic and Knockout Animals

The present invention provides transgenic and knockout animals that develop ovarian tumors and accurately recapitulate many of the features of the human ovarian tumor. Animal models of ovarian carcinoma are currently not available. Without limitation, particularly preferred transgenic or knockout animals are those in which the tumorigenic phenotype is fully penetrant, the rate of progression of the neoplasm is rapid, and/or the lifespan of the transgenic or knock-out animal is not shortened by a knockout- or transgene-related pathology in other organs. Of course, it will be appreciated that these traits are not required.

The generation of transgenic or knockout mice may provide a valuable tool for the investigation of human ovarian cancer by generating a mouse model for studying the disease, based on the description of the human Sal2 gene provided above. In one desirable embodiment, the hSal2 gene is used to produce the transgenic mice or the mSal2 gene is the target of the knockout. However, other Sal2 genes may also be used to produce transgenic mice provided that they are compatible with the mouse genome and that the protein encoded by this gene is able to carry out the function of the mSal2 protein.

Furthermore, a transgene, such as a mutant Sal2 gene, may be conditionally expressed (e.g., in a tetracycline sensitive manner). For example, the promoter for the Sal2 gene may contain a sequence that is regulated tetracycline and expression of the Sal2 gene product ceases when tetracycline is administered to the mouse. In this example, a tetracycline-binding operator, tetO, is regulated by the addition of tetracycline, or an analog thereof, to the organism's water or diet. The tetO may be operably-linked to a coding region, for example a mutant Sal2 gene. The system also may include a tetracycline transactivator (tTA), which contains a DNA binding domain that is capable of binding the tetO as well as a polypeptide capable of repressing transcription from the tetO (e.g., the tetracycline repressor (tetR)), and may be further coupled to a transcriptional activation domain (e.g., VP16). When the tTA binds to the tetO sequences, in the absence of tetracycline, transcription of the target gene is activated. However, binding of tetracycline to the tTA prevents activation. Thus, a gene operably-linked to a tetO is expressed in the absence of tetracycline and is repressed in its presence. The tetracycline regulatable system is well known to those skilled in the art and is described in, for example, WO 94/29442, WO 96/40892, WO 96/01313, and Yamamoto et al. (Cell 101:57-66 (2000).

In addition, the knockout organism may be a conditional knockout. For example, FRT sequences may be introduced into the organism so that they flank the gene of interest. Transient or continuous expression of the FLP protein may then be used to induce site-directed recombination, resulting in the excision of the gene of interest. The use of the FLP/FRT system is well established in the art and is described in, for example, U.S. Pat. No. 5,527,695, and in Lyznik et al. (Nucleic Acid Research 24:3784-3789(1996)).

Conditional knockout organisms may also be produced using the Cre-lox recombination system. Cre is an enzyme that excises DNA between two recognition sites termed loxP. The cre transgene may be under the control of an inducible, developmentally regulated, tissue specific, or cell-type specific promoter. In the presence of Cre, the gene, for example a Sal2 gene, flanked by loxP sites is excised, generating a knockout. This system is described, for example, in Kilby et al. (Trends in Genetics 9:413-421 (1993)).

Particularly preferred is a mouse model for ovarian cancer wherein the nucleic acid encoding a Sal2 gene, for example a mutant human Sal2 gene, is expressed in the cells of the ovary of the transgenic mouse such that the transgenic mouse develops ovarian tumors. The mice may contain a large T antigen transgene, such as one expressing an appropriate (carboxyl-terminal) fragment of large T antigen under the control of an ovarian specific promoter, or have a knockout of the mSal2 gene. In addition, ovarian cell lines from these mice may be established by methods standard in the art.

Transgenic animals may be made using standard techniques. For example, a gene encoding a cellular proto-oncogene, tumor suppressor gene, or other cellular protein, e.g., a cell cycle regulating protein, may be provided using endogenous control sequences or using constitutive, tissue-specific, or inducible regulatory sequences. Any tissue specific promoter may direct the expression of any Sal2 protein used in the invention, such as ovarian specific promoters, bladder specific promoters, and colon specific promoters. For example, knockout mutations may be engineered in the gene encoding the proto-oncogene or tumor suppressor gene and the mutated gene may be used to replace the wild-type Sal2 gene.

Construction of transgenes can be accomplished using any suitable genetic engineering technique, such as those described in Sambrook et al. (Molecular Cloning: A Laboratory Manual, Cold Spring Harbor Laboratory, N.Y., (1989)). Many techniques of transgene construction and of expression constructs for transfection or transformation in general are known and may be used for the disclosed constructs. Although the use of hSal2 in the transgene constructs is used as an example, any other protein encoded by an oncogene may also be used.

One skilled in the art will appreciate that a promoter is chosen that directs expression of the oncogene in the tissue in which cancer is expected to develop. For example, as noted above, any promoter that promotes expression of mSal2 in ovarian cancer cells can be used in the expression constructs of the present invention. Preferred ovarian promoters include, for example, promoters that are expressed in ovarian epithelial cells, such as, the polyoma virus promoter, the SPARK promoter, and the DOC-2 promoter. One skilled in the art would be aware that the modular nature of transcriptional regulatory elements and the absence of position-dependence of the function of some regulatory elements, such as enhancers, make modifications such as, for example, rearrangements, deletions of some elements or extraneous sequences, and insertion of heterologous elements possible. Numerous techniques are available for dissecting the regulatory elements of genes to determine their location and function. Such information can be used to direct modification of the elements, if desired. It is preferred, however, that an intact region of the transcriptional regulatory elements of a gene is used. Once a suitable transgene construct has been made, any suitable technique for introducing this construct into embryonic cells can be used, an example of such a technique is provided in Example 9.

Animals suitable for transgenic experiments can be obtained from standard commercial sources such as Taconic (Germantown, N.Y.). Many strains are suitable, but Swiss Webster (Taconic) female mice are preferred for embryo retrieval and transfer. B6D2F (Taconic) males can be used for mating and vasectomized Swiss Webster studs can be used to stimulate pseudopregnancy. Vasectomized mice and rats are publicly available from the above-mentioned suppliers. However, one skilled in the art would also know how to make a transgenic mouse or rat. An example of a protocol that can be used to produce a transgenic animal is provided in Example 9.

Use of Transgenic and Knockout Animals

The disclosed transgenic and knock-out animals may be used as research tools to determine genetic and physiological features of a cancer, and for identifying compounds that can affect ovarian and other tumors. Knockout animals also include animals where the normal gene has been inactivated or removed and replaced with a polymorphic allele of this gene. These animals can serve as a model system for the risk of acquiring a proliferative disease that is associated with a particular allele.

In general, the method of identifying markers associated with a proliferative disorder, such as ovarian tumors, involves comparing the presence, absence, or level of expression of genes, either at the RNA level or at the protein level, in tissue from a transgenic or knock-out animal as described above, and tissue from a matching non-transgenic or knock-out animal. Standard techniques for detecting RNA expression, e.g., by Northern blotting, or protein expression, e.g., by Western blotting, are well known in the art. Differences between animals such as the presence, absence, or level of expression of a gene indicate that the expression of the gene is a marker associated with a proliferative disorder, such as ovarian tumors. The molecular markers, once identified, can be used to predict whether patients with carcinoma will have indolent or aggressive disease, and may be mediators of disease progression. Identification of such mediators would be useful since they are possible therapeutic targets. Identification of markers can take several forms.

One method by which molecular markers may be identified is by use of directed screens. Patterns of accumulation of a variety of molecules that may regulate growth can be surveyed using immunohistochemical methods. Screens directed at analyzing expression of specific genes or groups of molecules implicated in pathogenesis can be continued during the life of the transgenic or knockout animal. Expression can be monitored by immunohistochemistry as well as by protein and RNA blotting techniques. Mestastatic foci, once formed, can also be subjected to such comparative surveys.

Alternatively, molecular markers may be identified using genomic screens. For example, ovarian tissue can be recovered from young transgenic or knockout animals (e.g, that may have early stage carcinoma) and older transgenic or knockout animals (e.g., that may have advanced stage carcinoma), and compared with similar material recovered from age-matched normal littermate controls to catalog genes that are induced or repressed as disease is initiated, and as disease progresses to its final stages. These surveys will generally include cellular populations in the ovary.

This analysis can also be extended to include an assessment of the effects of various treatment paradigms (including the use of compounds identified as affecting ovarian tumors in the transgenic or knockout animals) on differential gene expression (DGE). The information derived from the surveys of DGE can ultimately be correlated with disease initiation and progression in the transgenic or knockout animals.

The following examples are meant to illustrate the invention and should not be construed as limiting.

### EXAMPLES

### Example 1

### Isolation of TMD-25 Using a ‘Tumor Host Range’ Selection

A procedure for isolating ‘tumor host range’ mutants (e.g., T-HR mutants) and identifying cellular targets is outlined in below.

### Identification of a Host Factor that Interacts with T Antigens

1) Select Host Range Mutants

2) Identify Host Range Mutations

3) Identify Host Range Target and Validation

4) Biological Properties:

(i) Viral DNA Replication

(ii) Transformation

(iii) Tumorigenicity

Permissive hosts were chosen based on a screen of mouse cell lines derived from non-polyoma-induced tumors or transformed cells using the following criteria: (i) susceptibility to lytic infection by wild-type polyoma virus, and (ii) ability to be used in standard plaque assays.

Among a number of qualifying cell lines, two were chosen: A6241, derived from a spontaneous mammary tumor in a C57BR mouse, and TCMK-1, a SV40-transformed baby mouse kidney cell line. Primary baby mouse kidney epithelial cells (BMK) were used throughout as the non-permissive host.

Randomly mutagenized virus was prepared by passage of a plasmid containing wild-type polyoma viral DNA through the error prone Mut D strain of E. coli, followed by excision of the viral genome and transfection into TCMK-1 cells. After several cycles of virus growth in the same cells, individual plaques were isolated using TCMK-1 cells. An aliquot of virus in each plaque suspension was inoculated into BMK cell cultures. Virus from plaques that induced no cytopathic effect (CPE) on BMK cells after 10-14 days was amplified using TCMK-1 cells. Mutant DNAs were cloned, reconstituted as virus by transfection of permissive cells, and confirmed to retain the desired host range. The frequency of mutants was approximately one in several thousand plaques tested. The T-HR mutant TMD-25 was isolated by this procedure.

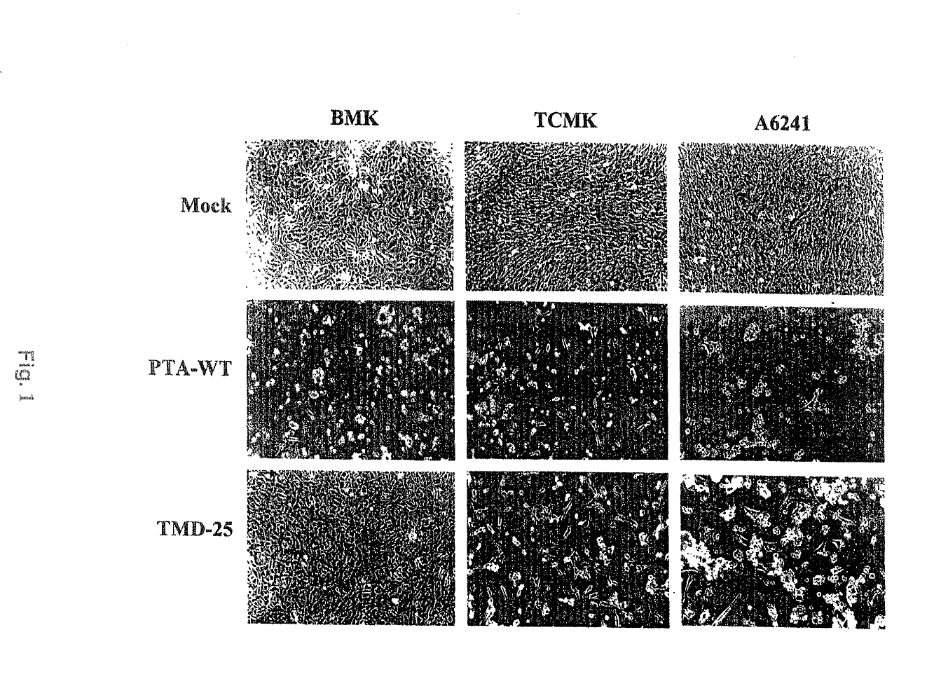

FIG. 1 shows the results of CPE tests comparing wild-type polyoma virus and TMD-25 growth in BMK, TCMK-1, and A6241 cells. Primary baby mouse kidney cells (BMK), SV40 Large T antigen transformed mouse kidney cells (TCMK), and spontaneous mouse mammary tumor cells (A6241) were mock-infected (Mock), or infected with 2-5 pfu of wild-type polyoma virus (PTA) or of T-HR mutant TMD25. The photographs were taken 14 days post infection and show the different cytopathic effect of viral growth.

TMD25 mutants grew poorly, if at all, on primary BMK cells, but could grow on transformed or tumor-derived cells, while wild-type polyoma virus grew well on all three cell-types. Extensive CPE developed in the TCMK-1 and A6241 cultures infected by the TMD25 mutant. Infectious mutant virus was produced in these cultures, although with somewhat slower kinetics and with lower final yields compared to wild-type virus. In contrast, no discernible CPE was noted in mutant-infected BMK cultures, even after extended periods of incubation of up to three weeks. Growth of TMD-25 on the spontaneous tumor line A6241 rules out the possibility that its growth depends strictly on complementation by SV40 large T antigen, which is expressed in TCMK-1.

### Example 2

### Sequencing of TMD-25 and Screening for Targets in Yeast

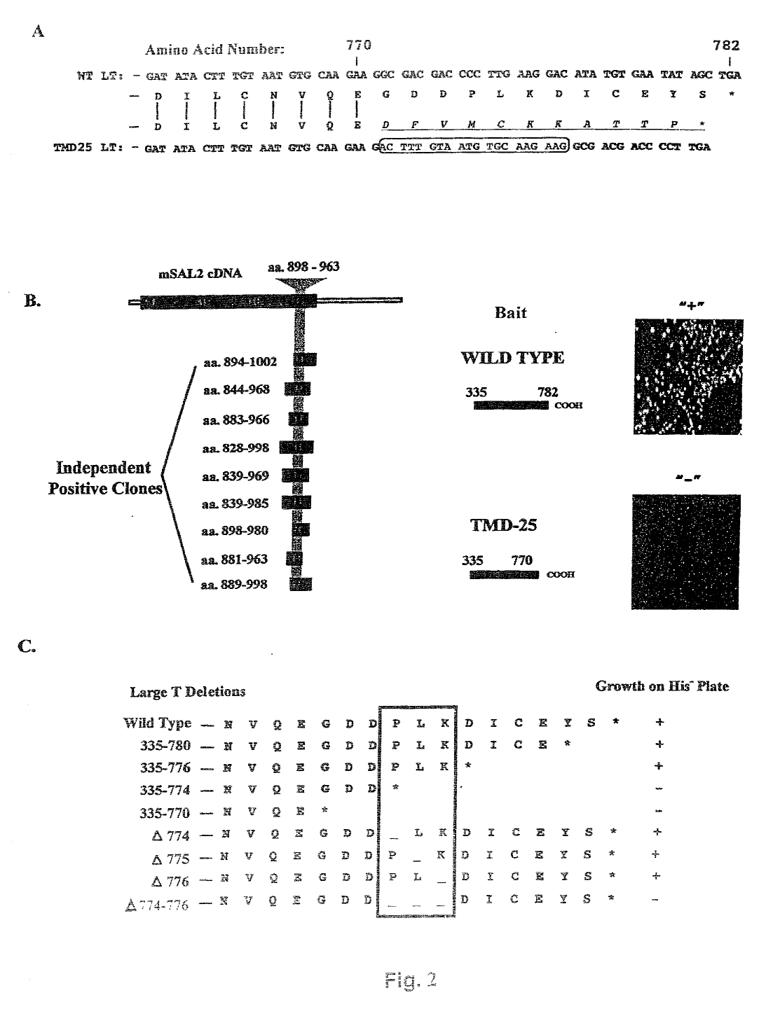

The mutation in TMD-25 responsible for its ‘tumor host range’ was localized to the carboxyl-terminal half of polyoma large T antigen as a result of studies using chimeric viruses constructed by ligating complementary DNA fragments from TMD-25 and wild-type virus. A combination of marker rescue and sequence analysis of this region revealed a twenty base pair duplication (circled) in TMD-25 encompassing the carboxyl-terminus of large T antigen. The resulting frameshift leads to replacement of the last 12 amino acids by 11 foreign residues (underlined) (SEQ ID NOS:9 to 12; FIG. 2A).

It is possible that the carboxyl-terminal region of large T antigen is involved in binding to some cellular target as an essential step in virus growth and that the mutation in TMD-25 abolishes this interaction. As a first step toward identifying a possible cellular target, a cDNA library constructed from 9.5 to 10.5 day-old mouse embryos was screened in yeast two-hybrid assays, using the carboxyl-terminal portion of normal large T antigen (amino acids 335-782) as bait.

Twenty-two positive clones were analyzed. Nineteen of these clones were represented by nine independent but overlapping cDNA sequences that centered around a sixty-six amino acid region (amino acids 900-965) encompassing a zinc finger pair in the carboxyl-terminal region of the mSal2 protein cDNAs (FIG. 2B, left panel, and discussed below). The identified sequences showed strong homology to the human gene hSal2, which is related to spalt in Drosophila.

The positive mSal2 clones did not interact with the carboxyl-terminus of TMD25 large T antigen, as indicated by the growth (+) of yeast colonies on histidine minus plates when using normal polyoma large T antigen as bait, but no growth using TMD25 large T antigen as bait (FIG. 2B, right panel), consistent with the notion that the host range defect of TMD-25 is based on its inability to bind this protein. All the His⁺ yeast colonies were also LacZ positive.

On continuous propagation in permissive cells, the TMD-25 mutant proved to be unstable, giving rise to wild-type virus revertants. To obtain a stable mutant and to further pinpoint the region of large T antigen essential for binding, (SEQ ID NOS:13 to 21), an analysis of the wild-type bait construct was carried out using mSal2 interaction in yeast as an assay (FIG. 2C). Truncation of the last six amino acids had no perceptible effect, but further truncations into the P-L-K sequence at positions 774-776 resulted in a loss of interaction. A deletion of these three amino acids in the context of an otherwise intact large T antigen was sufficient to prevent interaction with mSal2 and to recreate the host range phenotype shown in FIG. 1. The large T antigen deletion mutant 774-776 is hereafter referred to as TMD-25. The original defect of TMD25 is underlined, and the three amino acid region is framed in FIG. 2.

### Example 3

### Validation of mSal2 as a Target of Large T Antigen

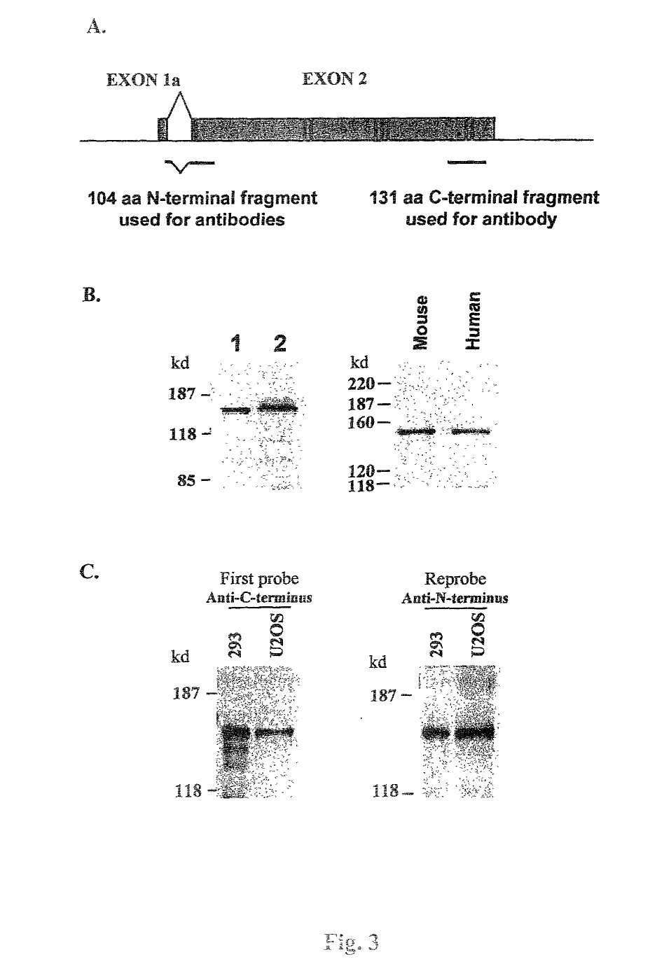

A complete cDNA was obtained using RACE (rapid amplification of cDNA ends). The sequence was found to be identical to that reported recently for mSal2, with a Glu rather than a Lys residue at position 350. The genomic sequence indicates two alternate short 5′ exons each encoding 24 amino acids and one unique 3′ exon encoding 980 amino acids. The overall homology with hSal2 is 85% using the BLAST 2 Sequence program. Eight Zinc fingers are apparent in exon 2. These zinc fingers are organized in four groups with the carboxyl-terminal pair presumed to be an essential part of the large T antigen interaction domain (FIGS. 2B and 3A). FIG. 3A shows the corresponding gene region of the mSal2 protein fragments used to develop antibodies. The exons are boxed, with the zinc fingers represented as stripes.

FIG. 3B shows the antibody detection of in vitro translated full-length mSal2 and p150^{sal2} in mouse and human cells. A polyclonal antibody was made in rabbits against a GST fusion protein containing 131 amino acids from the carboxyl-terminal large T antigen interaction domain. Extracts of mouse 624 and human 293 cell lines probed with this antibody show a single protein species migrating at approximately 150 kDa (FIG. 3B, Right Panel). A monoclonal antibody against a 108 amino acid amino terminal fragment spanning exons 1 and 2 was isolated (FIG. 3A). This antibody also detected mSal2 as a 150 kDa protein as an in vitro translation product (Tr), as well as a protein present in normal mouse brain extracts (Br)(FIG. 3B). This gene product of mouse and human origin is referred to as p150^{sal2}. To confirm that the single band from the human cell extract is hSal2, extracts from two human cell lines were first probed with the polyclonal antibody made against the carboxyl-terminus of mSal2. The filter was then stripped and reprobed with the anti-mSal2 amino-terminus polyclonal antibody. The identical band was detected with each of the two antibodies in the human cell lysates (FIG. 3C).

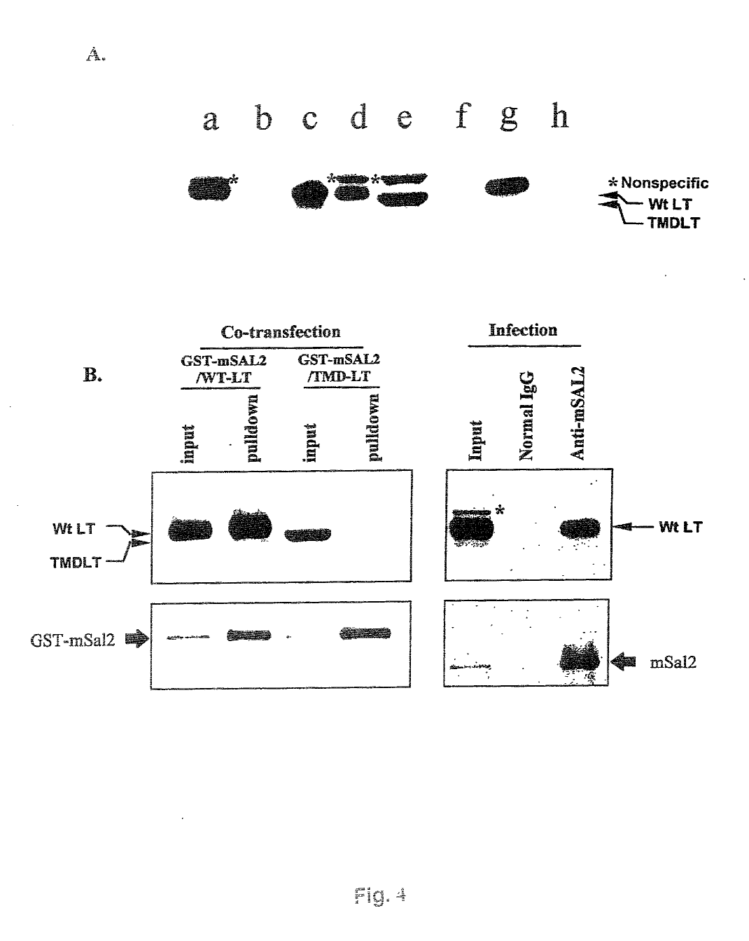

In vitro pull-down assays were carried out using a GST fusion of the large T antigen interaction domain of p150^{sal2} and extracts of lytically infected or transfected cells (FIG. 4A). The filter was blotted with an anti-large T antigen antibody. Lanes “a” to “c” show pulldown assays using wild-type polyoma, lytic infected BMK cells: lane “a” shows input extract from normal (WT) Py infected BMK cells; lane “b” shows cell extract from lane “a” pulled down with GST alone; lane “c” shows cell extract from lane “a” pulled down with GST-mSal2 fusion protein. Lanes “d” to “h” show pulldown assays using cell extracts of 3T3 cells transfected with WT large T antigen or TMD25 large T antigen cDNA: lane “d” shows the input extract from 3T3 transfected with WT large T antigen cDNA; lane “e” shows the input extract from 3T3 transfected with TMD25 large T antigen cDNA; lane “f” shows the extract of WT large T antigen cDNA transfected 3T3 cells pulled down with GST alone; lane “g” shows the extract of WT large T antigen cDNA transfected 3T3 cells pulled down with GST-mSal2 fusion protein; and lane “h” shows the extract of TMD25 large T antigen cDNA transfected 3T3 cells pulled down with GST-mSal2 fusion protein. Normal large T antigen synthesized during infection of BMK efficiently binds the GST-mSal2 fragment (lanes a to c). Comparing extracts of 3T3 cells transfected with either wild-type, or TMD-25, large T antigen cDNAs only the wild-type shows binding (lanes d to g).

To confirm the large T-p150^{sal2} interaction in vivo, 3T3 cells were doubly transfected with a vector expressing full length GST-mSal2 and either wild-type, or TMD-25 mutant, large T antigen cDNAs (FIG. 4B Left Panel). Cell extracts were pulled down with glutathione beads. After electrophoresis and transfer, the filter was blotted with anti-large T antigen antibody to show the binding of WT or mutant large T antigen. The same filter was blotted again with a monoclonal antibody against mSal2 to show that the level of expression of GST-mSal2 is similar in both the WT large T antigen and the TMD25 large T antigen experiments. Each lane is labeled and the input equaled 3% of the extracts used in the co-precipitation assay. Complexes containing normal large T antigen were readily recovered, but no evidence of binding was seen with the mutant large T antigen.

A further experiment was done to confirm the interaction between the large T protein and p150^{sal2} during a lytic viral infection. An extract of wild-type virus-infected BMK cells was prepared 24 hours post-infection and incubated with polyclonal serum made against the amino terminal mSal2 fragment. The anti-mSal2 immunoprecipitate was separated and blotted with an anti-T monoclonal antibody. Some of the large T antigen present in the virus-infected cell extract clearly immunoprecipitated with mSal2, showing that these two proteins interact (FIG. 4B Right Panel). Polyoma large T and p150^{sal2} most likely interact directly through their carboxyl-terminal regions, although additional factors may be involved in mediating the binding.

### Example 4

### TMD-25 is Defective in Virus Growth and Tumor Induction in the Newborn Mouse

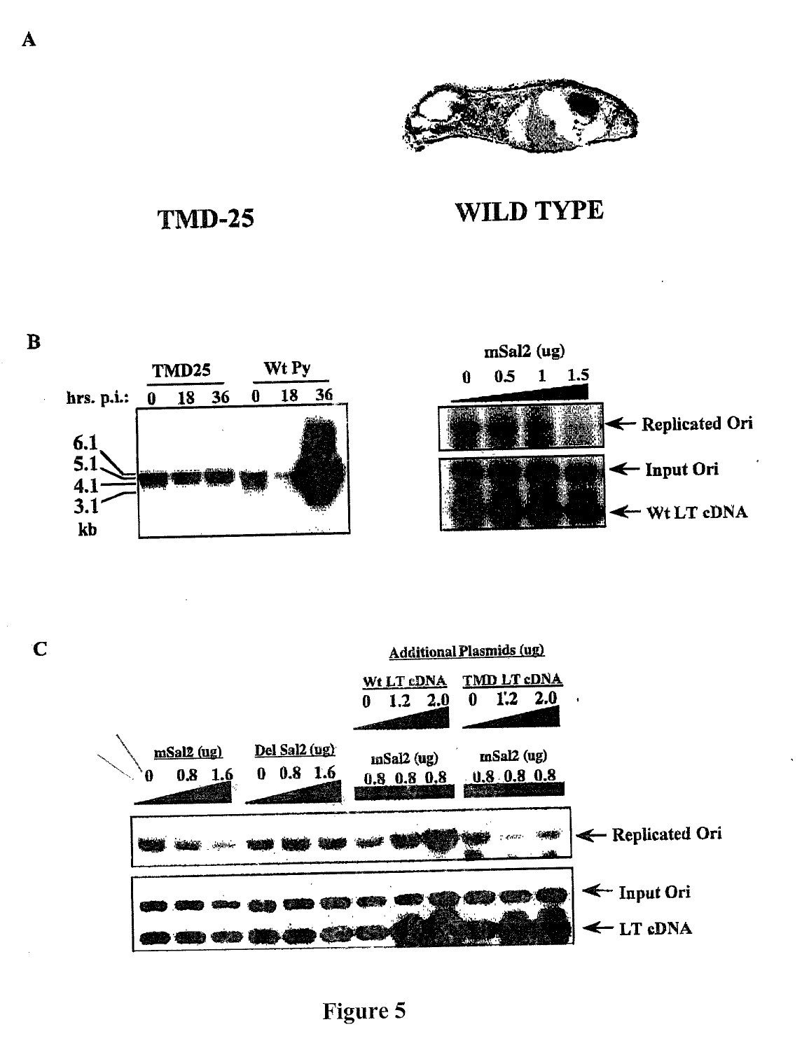

Newborn mice were inoculated with either wild-type or TMD-25 mutant virus and followed for development of tumors. The ability of TMD-25 to replicate and spread in the newborn mouse was examined by whole mouse section hybridization (Dubensky et al., J. Virol. 65:342-349 (1991). At ten days post inoculation the mutant showed no signs of replication and spread while the wild-type virus established a disseminated infection with extensive replication in many tissues (FIG. 5A).

Tests for virus replication were carried out on ten-day old animals by whole mouse section hybridization using a ³⁵S-labelled viral DNA probe (FIG. 5A). Newborn mice were inoculated subcutaneously with TMD25 or PTA (1×10⁶ each) and sacrificed ten days later. Frozen sections were probed with ³⁵S labeled viral DNA with overnight exposure. Wild-type PTA showed strong replication in kidney, skin, and bones, while the TMD25 mutant showed no sign of viral replication in any of the organs. Table 2 shows a comparison of tumor induction profile between mSal2 binding mutant TMD25 and wild-type PTA viruses. Newborn mice were inoculated as described above, and sacrificed five months later. Pathological examinations were performed for tumor profile. Wild-type virus rapidly established a disseminated infection and induced a broad spectrum of tumors (Table 2). In contrast, TMD-25 failed to replicate and spread. The only tumors found in mutant-infected mice were subcutaneous fibrosarcomas and these developed only at the site of virus inoculation. Since TMD-25 is defective in replication but retains normal middle and small T functions, these findings are consistent with the expectation that the input mutant virus would be able to infect and transform cells locally but be unable to spread.

These findings are consistent with the expectation that TMD-25 would retain wild-type transforming ability based on its retention of normal middle and small T functions, yet be unable to induce a broad spectrum of tumors because of a failure to replicate and spread. Direct tests of the mutant's transforming ability were carried out using standard assays with an established line of rat embryo fibroblasts (Dahl et al., Mol. Cell Biol. 16:2728-2735 (1996)). Transformation of these cells does not depend on virus replication, and middle T alone suffices for transformation (Raptis et al., Mol. Cell Biol. 5:2476-2485 (1985)). Mutant virus-infected cells gave rise to foci resembling those induced by wild-type virus; cells derived from one such focus were confirmed, by DNA sequencing, to carry the mutant viral genome. Using DNA transfection followed by measuring colony formation in soft agar, transforming efficiencies were found to be essentially identical for wild-type and mutant viral DNAs—approximately 10-20 colonies/10⁵ cell/μg viral DNA. The failure of TMD-25 to induce tumors at sites distant from the site of inoculation is therefore not due to any defect in transforming ability, but rather to its inability to replicate and establish a disseminated infection.

To investigate whether binding of p150^{sal2} by large T antigen is necessary for viral DNA replication, low molecular weight DNA from BMK cells infected by wild-type or mutant virus was extracted and analyzed by Southern hybridization. The results show clearly that the mutant was unable to replicate its DNA in the non-permissive host (BMK) cells 36 hr post infection (FIG. 5B, Left Panel). BMK cells were infected with TMD25 and wild-type virus (Wt Py). Low molecular weight DNA was isolated at 0, 18, 36 hrs post infection (p.i.) for Southern blot with virus DNA probe. These results suggest that p150^{sal2} can act, directly or indirectly, to inhibit viral DNA replication.

Furthermore, when over expressed in normal 3T3 cells, p150^{sal2} inhibited wild-type viral DNA replication in a dose-dependent manner (FIG. 5B, right panel). Polyoma origin clone pUCori (Ori) and large T -expressing plasmid, (Wt LT cDNA), were co-transfected with increasing amount of plasmid expressing mSal2. Newly replicated DNA was detected with origin specific probe (top). The filter was striped and re-probed with LT and origin specific probe to show that similar amount of origin and LT DNA were present in each transfection. Similarly, in additional origin replication assays we showed that, when the amount of mSal2 is held constant, an increasing amount of wild-type large T antigen, but not TMD25 large T antigen abolishes mSal2 inhibition of polyoma virus replication (FIG. 5C). These results show that p150^{sal2} imposes a block to viral DNA replication and that the block can be overcome by wild-type large T antigen. Accordingly, p150^{sal2} proteins and nucleic acids may be used as anti-viral agents.

### Example 5

### Expression Pattern of p150^{sal2} in the Mouse

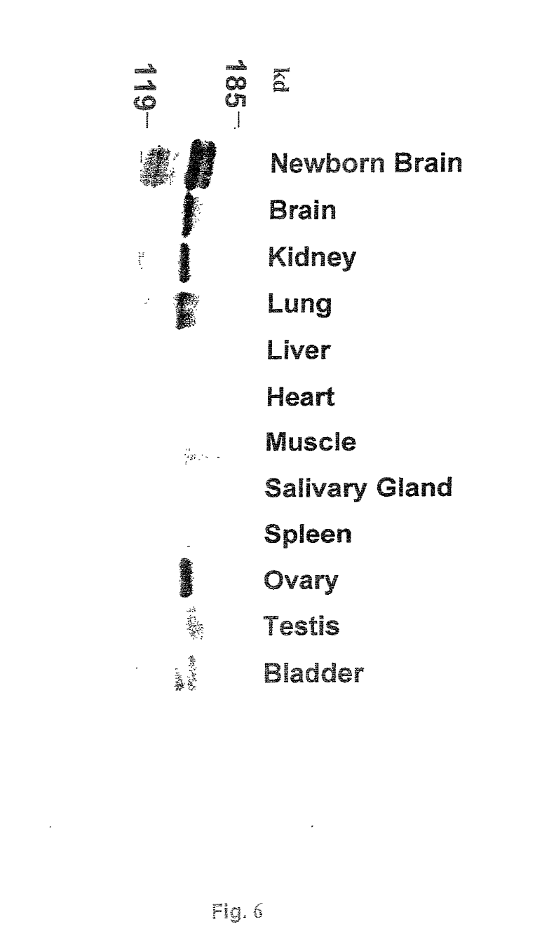

Normal mouse tissues were extracted and tested for expression of p150^{sal2} by Western blot (FIG. 6). Tissues from ten to twelve-day old mice were dissected and extracted in NP-40 lysis buffer. 200 μg of protein from various tissues were loaded onto each lane as labeled. The proteins were detected using a monoclonal antibody against the amino-terminus of mSal2. Tissue from brain, kidney, lung, bladder and uterus clearly shows expression of the protein, while tissue from liver, skeletal muscle, spleen, salivary gland and heart was either negative or low in expression. These results are consistent with those reported earlier by Northern analysis. The finding that the kidney and lung are sites of strong expression is also consistent with the natural history of transmission of polyoma, which is thought to infect through the lung and amplify primarily in the kidney. Successful growth in these tissues would require the virus to be able to overcome any block to replication imposed by mSal2. TMD-25 fails to replicate its DNA in normal mouse cells, and overexpression of mSal2 blocks normal viral DNA replication.

### Example 6

### Expression of hSal2 in Human Ovarian Tumors

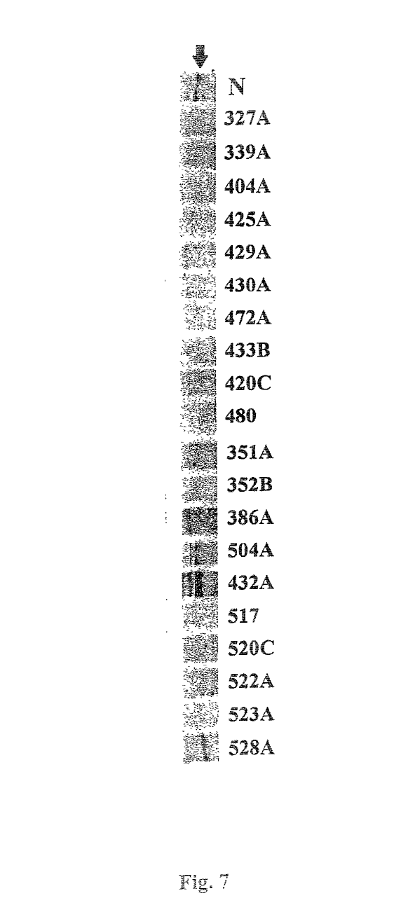

The hSal2 gene has been mapped to chromosome 14q12 but was not recognized initially as a tumor suppressor gene. It was subsequently shown by others that this region of 14q is associated with a loss of homozygosity in 49% of ovarian cancers (Bandera et al., supra) and about 25% of bladder cancers (Chang et al., supra). These findings, along with the underlying rationale of ‘tumor host range’ selection, suggest the possibility that sal2 may function as a tumor suppressor. To test this possibility more directly, a screen for p150^{sal2} expression was carried out on extracts of ovarian carcinomas (FIG. 7). FIG. 7 shows a Western blot of human ovarian tumors. The expression level of p150^{sal2} in 20 ovarian carcinomas was compared with that of normal ovarian epithelial cells (N) in two panels. Fifty micrograms of protein were loaded in each lane and blotted with polyclonal antibody against p150^{sal2}. Each ovarian carcinoma was labeled by its case number. Arrows indicate the normal position of p150. A polyclonal anti-p150 antibody made against the mouse protein clearly recognizes the human protein (FIG. 3B above). A band of the same apparent molecular weight is seen in extracts of normal human ovarian epithelial cells (‘HOSE’).

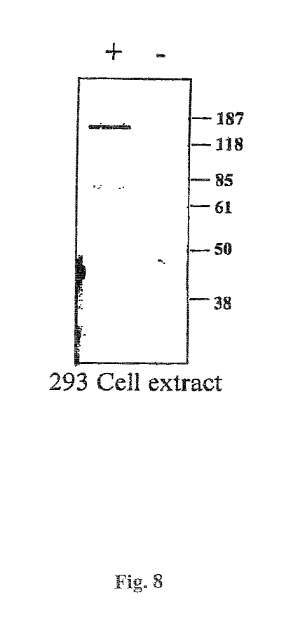

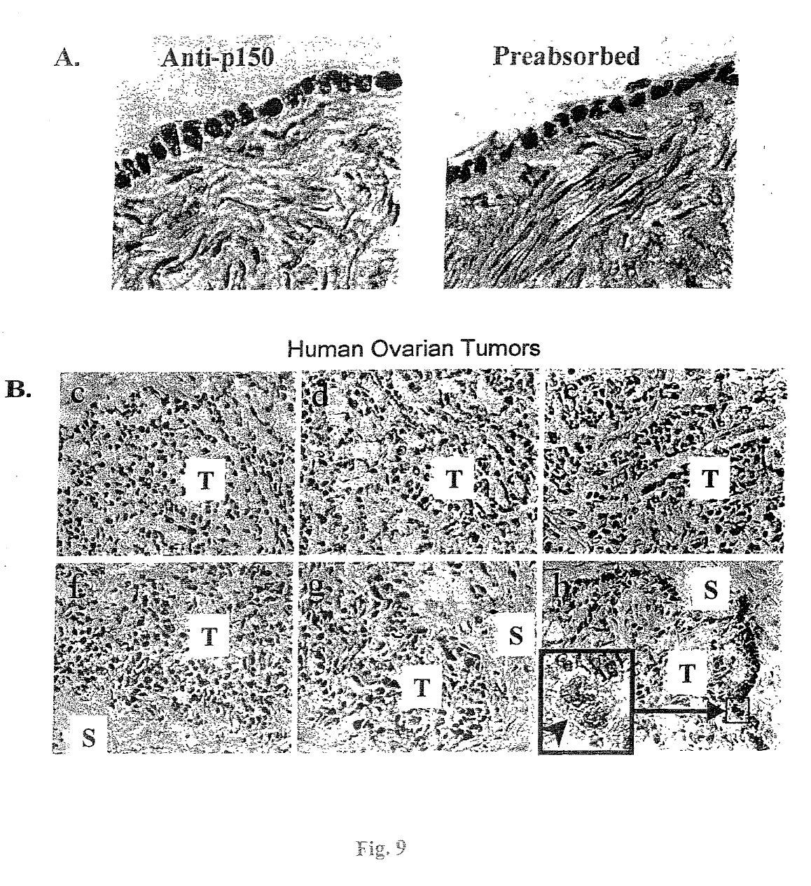

In situ staining with anti-p150 was carried out on frozen sections of normal ovary and several ovarian carcinomas, as well as in human 293 cells. FIG. 8 shows expression of p150^{sal2} in human 293 cells. A polyclonal antibody, HM867, raised against mSal2 carboxyl-terminus, was used to detect human p150^{sal2} in human 293 cells (lane +). As a negative control, the same protein extract was blotted with HM867 antibody that had first been depleted by incubation with the same antigen used to raise it (lane −). As a further example, FIG. 9 also shows immunostaining of p150^{sal2} in the human ovary and in ovarian tumors. FIG. 9A shows immunostaining of normal human ovarian tissue with a polyclonal serum preadsorbed with mSal2 protein. In the left-hand panel, normal human ovarian tissue is stained with a polyclonal serum preadsorbed with p150^{sal2}. In the right-hand panel, normal ovarian tissue is stained with polyclonal serum against p150^{sal2}. FIG. 9B shows six ovarian carcinoma tissue samples that were stained for p150^{sal2} (c thru h), where “T” stands for tumor cells and “S” stands for stromal cells. The insert in “h” shows cytoplasmic staining for p150^{sal2}. The nuclear staining of normal epithelial cells is readily apparent, but in the ovarian tumor cells the staining is reduced or cytoplasmic.

### Example 7

### A Point Mutation S73C in Human Sal2 is Present in Some Ovarian Tumors

DNAs from twenty-one ovarian carcinomas were digested and analysed by Southern hybridization using a probe of hSal2 coding sequences. hSal1 sequences were used as an unlinked internal control. No evidence of loss or gross rearrangement of the hSal2 locus was seen in any of the tumors examined. Deletions of 1 kb or less would not have been detected. The absence of p150^{sal2} expression in a majority of ovarian cancers may reflect mechanisms other than loss of the hSal2 gene itself, such as silencing of expression through promoter methylation, alterations in an upstream regulatory factor, or factors leading to instability of the protein itself.

To test for small mutations, DNAs from four tumors were extracted and the entire hSal2 coding regions sequenced on both strands. Two tumors from the panel shown in FIG. 7A that were positive for p150^{sal2} expression and two that were negative were chosen. The two negative tumors 327 and 523 showed no changes when compared to the controls and all showed sequences identical to the published genomic sequence (Genbank AE000658 and AE000521; Boysen et al, Genome Res. 330:330-338 (1997)). The two p150^{sal2}-positive tumors each showed a cysteine (TGT) substitution for serine (TCT) at position 73 (position 73 of SEQ ID NO:1), based on the first methionine in exon 1a (Kohlhase et al., Mamm Genome 11:64-69 (2000). The sequencing results showed only TGT in tumor 432 and a mixture of TGT and TCT in tumor 528. The serine codon TCT has been found at this position in all normal DNAs sequenced thus far (Kohlhase et al., Genomics 38:291-298 (1996); Boysen et al., Genome Res. 330:330-338 (1997)), indicating that ‘73S’ is a frequent normal allele. To know whether the S73C substitution represents a somatic mutation or germ line polymorphism, normal DNA from case 432 was sequenced. The result showed only TGT at codon 73, indicating that the hSal2 allele encoding cysteine represents a germ line polymorphism in this individual. DNAs from six ovarian carcinoma cell lines were also sequenced. One showed the same S73C substitution as seen in case 432 and another a G744R substitution.

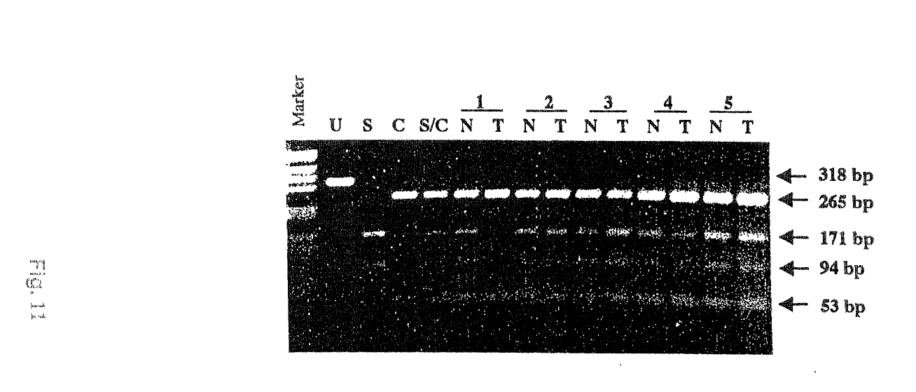

An example of the loss of the 73S allele is shown in FIG. 11. For this experiment, DNA was isolated from matched normal and ovarian tumor tissues. The 73S and 73C alleles were distinguished by PCR amplification and subsequent Mbo II digestion of a 318 bp product covering the region containing amino acid 73. In addition to a common Mbo II site (used to monitor the digestion status), this region contains another Mbo II site for the 73S allele, but not for the 73C allele (this is the discriminating Mbo II recognition site). Complete digestion of 73S allele by Mbo II produced three fragments (171 bp, 94 bp and 53 bp) while 73C allele produced two fragments (256 bp and 53 bp fragments—indicated by arrows). These fragments were resolved by electrophoresis on a 2% agarose gel. Although it is difficult to avoid the existence of normal tissue in the tumor used to isolate DNA, the intensity of the 73S bands (171 bp and 94 bp) is largely reduced indicating the loss of 73S allele (patient number 1). In this figure, “U” indicates undigested amplification product, “S” indicates a 73S homozygote control, “C” indicates a 73C homozygote control, and “S/C” indicates a 73S/C heterozygote control. The respective identification number of ovarian tumor patients is shown on top of their matched normal “N” and tumor “T” DNA.

### Example 8

### mSal2 Suppresses Growth of Ovarian Carcinoma Cells

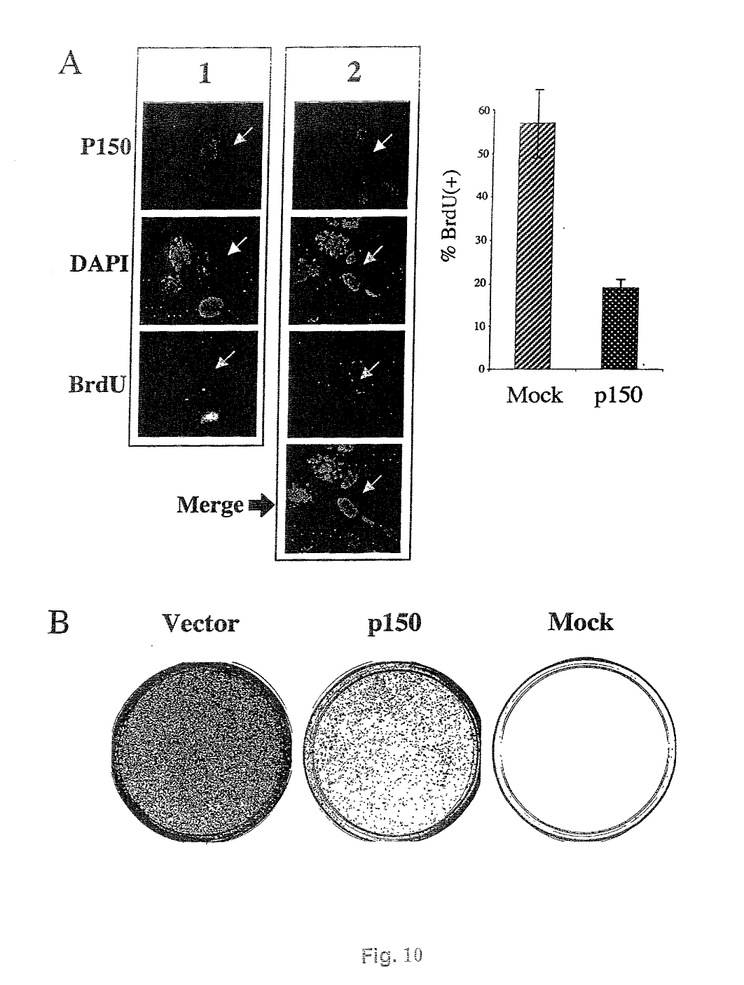

To characterize the biological function of Sal2, the ovarian carcinoma cell line SKOV3 was transfected with an mSal2 expression vector. SKOV3 cells were transfected with pcDNA-mSal2 (P150) or pcDNA3 vector (Mock), incubated in 0.5% serum for 48 hours, then in 15% serum and 100 μM BrdU for 20 hours. This cell line expresses little or no p150 as is indicated by Western analysis. Cells were examined by BrdU incorporation for DNA synthesis, for p150 expression, and for DAPI staining (FIG. 10A). The percent of cells in S-phase decreased from 57% in the control to 19% in cells expressing p150. In addition, 30-50% of cells expressing p150 appeared to be apoptotic as judged by DAPI staining compared to less than 10% of control cells. Arrows in frame 1 of FIG. 10A indicate a cell expressing p150 that is BrdU-negative. Arrows in frame 2 of FIG. 10A indicate an apoptotic cell expressing p150 with fragmented nuclear bodies as shown in the merged image. The bar graph in FIG. 10A shows the percentage of BrdU-positive cells in Mock and P150 expressing cells. In a colony reduction assay conducted over 14 days, a clear reduction in viable SKOV3 cells was seen in cells transfected with the expression vector, reflecting both growth suppressive, and apoptosis inducing activity of p150^{sal2} (FIG. 10B). Similar efficiencies of transfection (approximately 20%) were confirmed by a co-transfected GFP expression plasmid.

### Example 9

### Experimental Procedures

Selection of Tumor Host-Range Mutants

Cell lines used as permissive hosts include TCMK-1 (Black et al., Proc. Soc. Exper. Biol. Med. 114:721-727 (1963)) purchased from ATCC) and A6241 (Lukacher et al., J. Exp. Med. 181:1683-1692 (1995); Velupillai et al., J. Virology 73: 10079-10085 (1999)) have been described. Primary baby mouse kidney cells (BMK) were used as the non-permissive host. The genome of polyoma virus strain PTA was digested at the single BamHI site and cloned into pBlueScript (Stratagene) to create PTAHI. PTAHI was amplified in the Mut D strain of E. coli (Schaaper et al., Proc. Natl. Acad. Sci. U.S.A. 85:8126-8130 (1998)) to accumulate mutations randomly throughout the viral genome.

Yeast Two-Hybrid Screening

The polyoma PTA large T antigen carboxyl-terminal fragment (amino acids 333-781) was cloned into pGBT9 (Clontech) to generate pGBT9ITC used as a “bait” to screen a 9.5 to 10.5 day-old whole mouse embryo cDNA library in pVP16 (Vojtek et al., Cell 75:205-214 (1993)). Transformation and selection were performed according to the recommendations from Clontech.

Generation of TMD25 with a Minimum Deletion

Large T antigen carboxyl-terminal deletions used in the yeast two-hybrid analysis were generated on pGBT9ITC using the TRANSFORMER site-directed in vitro mutagenesis kit (Promega) according to manufacturer's recommendations.

Cloning of Full Length mSal2 cDNA

A complete cDNA sequence for mSal2 was obtained by RACE (Frohman) using the MARATHON cDNA amplification kit (Clontech) and RT-PCR products from BMK cells.

RFLP Test to Identify a Polymorphism in Sal2

Amino acid 73 of human p150^{sal2} is polymorphic. This amino acid may be a serine encoded by the codon TCT (73S) or a cysteine encoded by the codon TGT (73C). The two alleles may be distinguished by PCR amplification of the genomic region encompassing the sequence encoding hSal2 amino acid 73 and digesting the PCR product using either the restriction enzyme MobII or EarI. These enzymes cut the DNA close to the codon encoding amino acid 73. The primers used to amplify the DNA prior to digestion with MobII were, 5′-CTTGTTAATTAGAGCCTCGGTATACC-3′ (SEQ ID NO:7) and 5′-GCACGGAGGACCCAGAATCTGG-3′ (SEQ ID NO:8).

The PCR cycle used was 98° C. for 2 minutes followed by 35 cycles of 94° C. for 1 minute, 55° C. for 1 minute, and 68° C. for 1 minute. The last PCR cycle was followed by incubating the reaction at 72° C. for 10 minutes. The PCR products were digested with MobII in a solution containing 5 μl PCR mixture, 2 μl enzyme buffer (10 fold concentrated), 12 μl water, and 1 μl MobII (5 units/μl). The restriction digest was performed at 37° C. for two hours followed by heating the reaction to 70° C. for twenty minutes prior to loading ten to twenty microliters of the mixture onto a 2% agarose gel. Five microliters of undigested PCR product are added to a control lane on the gel. The exprected size of the uncut PCR product is 318 bp. The expected MobII restriction fragments for the 73S allele are 171, 94, and 53 bp and the expected Mob II restriction fragments for the 73C allele are 265 and 53 bp. A mixture of the 73S and 73C alleles would be expected to yield fragments of 265, 171, 94, and 53 bp. The 53 bp fragment is common to both alleles and may be used to monitor the digestion status in order to distinguish between heterozygotes and an incomplete digestion.

In Vitro GST Pull-Down Assay

Full-length polyoma normal large T antigen cDNA and TMD25 large T antigen cDNA were cloned into pcDNA3 to create CMVLT and CMVTMDLT respectively. The mSal2 fragment (amino acids 841-971), containing the last zinc finger pair, was cloned into pGEX4T1 (Pharmacia) to generate GST-mSal2 fusion protein in E. coli. The fusion protein was bound to glutathione-Sepharose 4B beads (purchased from Pharmacia) according to the manufacturer's instructions. For the association of GST-mSal2 fusion with large T antigen, BMK cells infected by PTA, or 3T3 cells transfected with wild-type or TMD25 large T antigen expression constructs CMVLT or CMVTMDLT, were extracted with NP-40 lysis buffer (pH 7.9) (Benjamin et al., Proc. Natl. Acad. Sci. U.S.A. 67:394-399 (1970)). 500μl of cell lysate were incubated with 50 μl of 50% GST-Sal2 or GST beads for 2 hours. After washing four times with PBS, the bound protein was subjected to Western blot analysis using monoclonal antibody F4, which recognizes T antigens (Dahl et al., Mol. Cell. Biol. 16:2728-2735 (1996)).

In Vivo GST Pull-Down Assay

The full-length mSal2 coding region was cloned into a eukaryotic GST fusion vector, pEBG (Luo et al., J. Biol. Chem. 270:23681-23687 (1995)) to generate the construct pEBGSAL. NIH 3T3 cells were co-transfected with pEBGSAL and CMVLT or CMVTNDLT in a ration of 1 to 1 using LIPOFECTAMINE 2000 (Gibco/BRL) following the manufacturer's protocol. The cells were harvested 48 hours post transfection. The lysate was centrifuged at 3,000 rpm and the supernatant was incubated with 50-100 μl glutathione-Sepharose 4B beads for 2 hours. The beads were washed four times with PBS containing 0.01% NP-40 and bound proteins were immunoblotted with the F4 antibody and an antibody against p150^{sal2} (Dahl et al., supra (1996)).

In Vivo Co-Immunoprecipitation of mSal2 and Polyoma Large T

Fifty microliters of 50% protein A beads (Pharmacia) were incubated with purified rabbit polyclonal anti-amino-terminal mSal2 antibody or normal rabbit IgG in 1 ml NP-40 lysis buffer at 4° C. for 2 hours. The beads were washed four times with PBS. BMK cells infected with wild-type virus were extracted 24 hours post infection. Two milligrams of total protein were incubated with either the anti-mSal2 or normal IgG beads in NP-40 lysis buffer containing 1% BSA for 2 hours at 4° C. The beads were washed four times with 0.1% Tween-20 in PBS and the proteins were separated by SDS-PAGE. Polyoma large T and mSal2 were detected using anti-T and anti-mSal2 monoclonal antibodies.

Viral DNA Replication Assays

Plasmid pUCori and the polyoma origin replication assay are described in Gjorup et al. (Proc. Natl. Acad. Sci. USA 91:12125-12129 (1994)). Cells were grown on 6 well plates and infected with virus or transfected with DNA. Low molecular weight DNA was isolated as described by Hirt (J. Mol. Biol. 26:365-369 (1967)). After purification, the DNAs were resuspended in 80 μls water. One to five micrograms of DNA were subjected to restriction digestion. For virus infection, the viral genome was first linearized with Eco R1. For transfection, pUCori and CMVLT were first digested with Dpn I and Hind III. The newly synthesized pUCori DNA is Dpn I resistant because of the lack of methylation in eukaryotic cells and the input plasmid DNA is sensitive to Dpn I digestion because of the E. coli methylation of the recognition site. The DNA fragment was resolved on a 1% agarose gel for Southern analysis using origin specific and LT specific probes.

Western Blots for Detection of p150^{sal2}

Tissue extracts were prepared from C3H/BiDa mice by homogenization in NP-40 lysis buffer (pH 7.9) and centrifugation at 8,000 rpm. Fifty micrograms of protein (Bio-Rad Assay) from each sample was separated by SDS-PAGE and blotted on nitrocellulose membranes. A monoclonal antibody against mSal2 was used to detect p150^{sal2}.

Stripping Western Filters for Reprobing

After first antibody probing, the used filter is incubated in stripping solution (50 mM Tris-Cl, pH 6.8, 2% SDS and 100 mM β-mercaptoethanol) for 30 minutes at 60° C. The filter is washed twice in PBS and tested for the absence of the previously used antibody by development and exposure to an X-ray film. This procedure ensures that the filter can be used again in subsequent Western analyses.

Analysis of Ovarian Carcinomas

Surgical samples of human ovarian tissue were obtained under a protocol approved by the Human Subjects Committee of the Brigham and Women's Hospital. Ovarian tumor tissues were pulverized in liquid nitrogen and lysed in a buffer (1% Triton X-100, 21 μg/ml aprotinin, 1 mM phenylmethylsulfonyl fluoride, 0.5 μg/ml leupeptin, 4.9 mM MgCl₂, and 1 mM vanadate in PBS). The MICRO BAC Protein Assay Kit (Pierce) was used for protein quantitation. Twenty-five micrograms of protein from each sample was separated on an SDS-polyacrylamide gel and blotted on nitrocellulose membranes. A rabbit polyclonal antiserum that cross-reacts with hSal2 was used to detect p150^{sal2}. Specifically, this antiserum was raised against a GST-mouse p150 fusion protein that was first purified using Affinity Pak Immobilized Protein A (Pierce) according to manufacturer's instructions followed by an incubation with GST saturated glutathione beads (Pharmacia) in PBS for 30 minutes to eliminate antibodies against GST. As a negative control, the purified antibody was preadsorbed with the GST-p150 fusion protein.

Frozen sections of normal or tumor samples were fixed in neutral formalin for 10 minutes and permeabilized in cold ethanol/acidic acid (3:1) for 15 min. After washing four times in PBS for 10 minutes each, the sections were antibody stained and processed using VECTASTAIN ABC kit (Vector Laboratories) following the manufacturer's instructions.

DNAs were extracted from human ovarian carcinomas and from primary cultures of ovarian epithelial cells obtained by scraping the surface of normal ovarian tissue. DNA from normal human foreskin was used as a control. The coding region with the 0.4 kb intron of hSal2 was amplified using the primer pair (5′-CCACAACCATGGCGAATCCGAG-3′) (SEQ ID NO:5) and (5′-GGTGATGGAAGGCGAACAGCCAGG-3′) (SEQ ID NO:6). Long range PCR was performed (98° C. 4 min, then 94° C. 1 min, 60° C. 1 min, 68° C. 4 min, for 35 cycles) and sequencing was carried out using the High Throughput Core of the Dana Farber-Harvard Cancer Center. The coding region was sequenced twice and additional sequencing of both strands was performed for regions with suspected mutations. The resulting sequence was compared with the published hSal2 cDNA sequence (Kohlhase et al., Genomics 38:291-298, 1996) and genomic sequence (Boysen et al., Genome Research 7:330-338, 1997).

BrdU Incorporation

SKOV3 cells were transfected with pcDNA-mSal and the pcDNA 3 vector using BRL LIPOFECTAMINE 2000 according to the manufacturer's recommendations. Five to seven hours post transfection the cells were fed with 0.5% calf serum. After 48 hours, the cells were incubated with a medium containing 15% calf serum with 100 mM BrdU for 20 to 24 hours. A monoclonal antibody against BrdU (Amersham) was used to detect the incorporation. The cells were fixed, permeabilised and stained according to Amersham's recommendations except that a purified rabbit polyclonal antibody against the mSal2 carboxyl-terminus was mixed with the BrdU antibody for the detection of both BrdU incorporation and p150 expression. Secondary antibodies (anti-mouse Rhodamine and anti-rabbit Oregon Green) were also mixed. Cells were examined under fluorescence microscopy in order to identify BrdU and p150 positive cells.

Colony Reduction Assay

SKOV3 cells were transfected with a pcDNA-mSal or a pcDNA3 vector in a 6 well plates using 2 μg of DNA each. To monitor the transfection efficiency, 0.5 μg of pEGFPN1 (Clontech) was added to the test DNA in a separate tube. Transfection was performed according to GIBCO/BRL's recommendations using LIPOFECTAMINE 2000. Twenty-four hours after the transfection, the cells were re-seeded in 10 cm plates with medium containing 600 μg/ml G418 (GIBCO/BRL) and 10% calf serum. The EGFP expression was also monitored at this time. The G418 containing medium (neomycin medium) was changed every 3 to 4 days until mock-transfected cells had died and neomycin resistant colonies became apparent.

Preparation of DNA for Microinjection

As but one example, DNA clones for microinjection are prepared by cleaving the DNA with enzymes appropriate for removing the bacterial plasmid sequences and subjecting the DNA fragments to electrophoresis on 1% agarose gels in TBE buffer (Sambrook et al., Molecular Cloning: A Laboratory Manual (Cold Spring Harbor Laboratory, Cold Spring Harbor, N.Y., 1989)). The DNA bands are visualized by staining with ethidium bromide and the band containing the desired DNA sequences is excised. The excised band is then placed in dialysis bags containing 0.3 M sodium acetate pH 7.0. The DNA is electroeluted into the dialysis bags, extracted with phenol/chloroform (1:1), and precipitated by the addition of two volumes of ethanol. The DNA is then redissolved in 1 ml of low salt buffer (0.2 M NaCl, 20 mM Tris, pH 7.4, and 1 mM EDTA) and purified on an ELUTIP-D™ (Schleicher and Schuell) column. The column is first primed with 3 ml of high salt buffer (1M NaCl, 20 mM Tris, pH 7.4, and 1 mM EDTA) followed by washing with 5 ml of low salt buffer. The DNA solutions are passed through the column three times to bind the DNA to the column matrix. After one wash with 3 mls of low salt buffer, the DNA is eluted with 0.4 ml of high salt buffer and precipitated by the addition of two volumes of ethanol. DNA concentrations are measured by absorption at 260 nm in a UV spectrophotometer. For microinjection, DNA concentrations are adjusted to 5 μg/ml in 5 mM Tris, pH 7.4 and 0.1 mM EDTA. Other methods for purification of DNA for microinjection are also described in Hogan et al., Manipulating the Mouse Embryo (Cold Spring Harbor Laboratory, Cold Spring Harbor, N.Y., (1986)); in Palmiter et al., Nature 300:611 (1982); in the Qiagenologist, Application Protocols, 3^{rd} edition, published by Qiagen, Inc., Chatsworth, Calif.; and in Sambrook et al., Molecular Cloning: A Laboratory Manual (Cold Spring Harbor Laboratory, Cold Spring Harbor, N.Y., 1989). The procedures for manipulation of the rodent embryo and for microinjection of DNA are described in detail in Hogan et al., Manipulating the Mouse Embryo (Cold Spring Harbor Laboratory, Cold Spring Harbor, N.Y., (1986)), the teachings of which are incorporated herein.

Animal Experiments

Whole mouse section hybridizations (Dubensky et al., J. Virol. 68:342-349 (1991)) and tumor profiles (Dawe et al., Am. J. Pathol. 127:243-261 (1987)) were performed as described in these references.

Production of Transgenic Mice and Rats

The following is but one preferred means of producing transgenic mice. This general protocol may be modified by those skilled in the art.

Female mice six weeks of age are induced to superovulate with a 5 IU injection (0.1 cc, IP) of pregnant mare serum gonadotropin (PMSG; Sigma) followed 48 hours later by a 5 IU injection (0.1 cc, IP) of human chorionic gonadotropin (hCG, Sigma). Females are placed together with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA, Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with humidified atmosphere at 5% CO₂, 95% air until the time of injection. Embryos can be implanted at the two-cell stage.

Randomly cycling adult female mice are paired with vasectomized males. Swiss Webster or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos are transferred. After the transferring the embryos, the incision is closed by two sutures.

The preferred procedure for generating transgenic rats is similar to that described above for mice (Hammer et al., Cell 63:1099-112 (1990). For example, thirty-day old female rats are given a subcutaneous injection of 20 IU of PMSG (0.1 cc) and 48 hours later each female placed with a proven, fertile male. At the same time, 40-80 day old females are placed in cages with vasectomized males. These will provide the foster mothers for embryo transfer. The next morning females are checked for vaginal plugs. Females who have mated with vasectomized males are held aside until the time of transfer. Donor females that have mated are sacrificed (CO₂ asphyxiation) and their oviducts removed, placed in DPBA (Dulbecco's phosphate buffered saline) with 0.5% BSA and the embryos collected. Cumulus cells surrounding the embryos are removed with hyaluronidase (1 mg/ml). The embryos are then washed and placed in EBSs (Earle's balanced salt solution) containing 0.5% BSA in a 37.5° C. incubator until the time of microinjection.

Once the embryos are injected, the live embryos are moved to DPBS for transfer into foster mothers. The foster mothers are anesthetized with ketamine (40 mg/kg, IP) and xulazine (5 mg/kg, IP). A dorsal midline incision is made through the skin and the ovary and oviduct are exposed by an incision through the muscle layer directly over the ovary. The ovarian bursa is torn, the embryos are picked up into the transfer pipet, and the tip of the transfer pipet is inserted into the infundibulum. Approximately 10 to 12 embryos are transferred into each rat oviduct through the infundibulum. The incision is then closed with sutures, and the foster mothers are housed singly.

Generation of Knockout Mice

The following is but one example for the generation of a knockout mouse and the protocol may be readily adapted or modified by those skilled in the art.

Embryonic stem cells (ES), for example, 10⁷ AB1 cells, may be electroporated with 25 μg targeting construct in 0.9 ml PBS using a Bio-Rad Gene Pulser (500 μF, 230 V). The cells may then be plated on one or two 10-cm plates containing a monolayer of irradiated STO feeder cells. Twenty-four hours later, they may be subjected to G418 selection (350 μg/ml, Gibco) for 9 days. Resistant clones may then be analyzed by Southern blotting after Hind III digestion, using a probe specific to the targeting construct. Positive clones are expanded and injected into C57BL/6 blastocysts. Male chimeras may be back-crossed to C57BL/6 females. Heterozygotes may be identified by Southern blotting and intercrossed to generate homozygotes.

The targeting construct may result in the disruption of the gene of interest, e.g., by insertion of a heterologous sequence containing stop codons, or the construct may be used to replace the wild-type gene with a mutant form of the same gene, e.g., a “knock-in.” Furthermore, the targeting construct may contain a sequence that allows for conditional expression of the gene of interest. For example, a sequence may be inserted into the gene of interest that results in the protein not being expressed in the presence of tetracycline. Such conditional expression of a gene is described in, for example, Yamamoto et al. (Cell 101:57-66 (2000)).

TABLE 2
Tumor profiles of mutant TMD-25 and wild-type PTA virus

| | TMD-25 | PTA¹ |
|---|---|---|
| Fraction of mice with tumors | 7/7 | 32/32 |
| Mean age at necropsy | 202 d. | 82 d. |
| Epithelial tumors: | | |
| Hair follicle | 0/7 | 32/32 |
| Thymus | 0/7 | 29/32 |
| Mammary gland | 0/7 | 16/32 |
| Salivary gland | 0/7 | 23/32 |
| Mesenchymal tumors: | | |
| Fibrosarcomas | 7/7² | 1/32 |
| Renal medulla | 0/0 | 7/32 |
| Bone | 0/0 | 6/32 |

¹Data on PTA is taken from Dawe et al. (Am. J. Pathol. 127:243-261, 1987).
²Subcutaneous fibrosarcomas were found only at the site of virus inoculation.

TABLE 3
Summary of p150^{sa12} expression in human ovarian carcinomas

| p150^{sa12} Status | Number of Cases | Percent |
|---|---|---|
| Positive | 6 | 30 |
| Negative | 10 | 50 |
| Altered* | 4 | 20 |

*Refers to the apparent size of the Sa12 protein, which is different from that of normal ovarian epithelial cells.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All references cited herein are hereby incorporated by reference.

We claim:

1. A method of decreasing proliferation of an abnormally proliferating cell, said method comprising the step of contacting said abnormally proliferating cell with a Sal2 nucleic acid sequence, wherein said contacting results in the expression of a Sal2 polypeptide having tumor suppressive activity in said abnormally proliferating cell.

2. The method of claim 1, wherein said Sal2 polypeptide comprises the amino acid sequence of SEQ ID NO:1 or 3.

3. The method of claim 1, wherein said abnormally proliferating cell has a proliferative disease-associated alteration in a Sal2 nucleic acid sequence.

4. The method of claim 3, wherein said proliferative disease-associated alteration comprises a Sal2 nucleic acid sequence that encodes a polypeptide that contains a substitution of a Cys for the Ser at position 73 of SEQ ID NO:1.

5. The method of claim 1, wherein said abnormally proliferating cell is an ovarian cell.

6. A method of decreasing DNA tumor virus replication and dissemination, said method comprising the step of contacting a cell infected with a DNA tumor virus with a Sal2 nucleic acid sequence, wherein said contacting results in the expression of a Sal2 polypeptide in said cell infected with said DNA tumor virus and prevents said DNA tumor virus from replicating and disseminating.

7. The method of claim 6, wherein said Sal2 polypeptide comprises the amino acid sequence provided in SEQ ID NO:1 or 3.

8. The method of claim 6, wherein said DNA tumor virus is selected from the group consisting of, simian virus 40, human polyoma virus, herpes virus, primate adenoviruses, parvovirus, and papilloma virus.

9. An isolated Sal2 nucleic acid sequence encoding a polypeptide that contains a substitution of a Cys for the Ser at position 73 of SEQ ID NO:1.

10. The Sal2 nucleic acid sequence of claim 9, wherein said Sal2 nucleic acid sequence is a human Sal2 nucleic acid sequence.


SEQUENCE LISTING (21 sequences)

SEQ ID NO: 1 - protein, 1005 aa, Homo Sapiens
MAHESERSSRLGVPAGEPAELGGDASEEDHPQVCAKCCAQFTDPTEFLAHQNACSTDPPVMVIIGGQENPNNSSASSEPRPEGHNNPQVMDTEHSNPPDSGSSVPTDPTWGPERRGEESSGHFLVAATGTAAGGGGGLILASPKLGATPLPPESTPAPPPPPPPPPPPGVGSGHLNIPLILEELRVLQQRQIHQMQMTEQICRQVLLLGSLGQTVGAPASPSELPGTGTASSTKPLLPLFSPIKPVQTSKTLASSSSSSSSSSGAETPKQAFFHLYHPLGSQHPFSAGGVGRSHKPTPAPSPALPGSTDQLIASPHLAFPSTTGLLAAQCLGAARGLEATASPGLLKPKNGSGELSYGEVMGPLEKPGGRHKCRFCAKVFGSDSALQIHLRSHTGERPYKCNVCGNRFTTRGNLKVHFHRHREKYPHVQMNPHPVPEHLDYVITSSGLPYGMSVPPEKAEEEAATPGGGVERKPLVASTTALSATESLTLLSTSAGTATAPGLPAFNKFVLMKAVEPKNKADENTPPGSEGSAISGVAESSTATLMQLSKLMTSLPSWALLTNHFKSTGSFPLPLCARALGASPSETSKLQQLVEKIDRQGAVAVTSAASGAPTTSAPAPSSSASSGPNQCVICLRVLSCPRALRLHYGQHGGERPFKCKVCGRAFSTRGNLRAHFVGHKASPAARAQNSCPICQKKFTNAVTLQQHVRMHLGGQIPNGGTALPEGGGAAQENGSEQSTVSGAGSFPQQQSQQPSPEEELSEEEEEEDEEEEEDVTDEDSLAGRGSESGGEKAISVRGDSEEASGAEEEVGTVAAAATAGKEMDSNEKTTQQSSLPPPPPPDSLDQPQPMEQGSSGVLGGKEEGGKPERSSSPASALTPEGEATSVTLVEELSLQEAMRKEPGESSSRKACEVCGQAFPSQAALEEHQKTHPKEGPLFTCVFCRQGFLERATLKKHMLLAHHQVQPFAPHGPQNIAALSLVPGCSPSITSTGLSPFPRKDDPTIP

SEQ ID NO: 2 - DNA, 16080 bp, Homo sapiens
atatcacaccccagctggctatgtaatcatgaaataaggagaaacacataaatatttggttaaaacacctttaatgatagagggaaagacactaatatctcccgtctgttcttgacattttactaggttaggaagctctggagcctacagcttgaggagaagccatcgttcaagtcagtcaatagcaaaaccctcactctctcctcctcagaactcctgttccaaatgatcctatgttaagagtaaatactacaactcattacaagacggagaggcagggaggacgccacctggagctgggactcttaagaaccagacaatgacaaagacacaagccccagcctacggataggcaaaatgggtaggggtcttgaaagaggaagataaggaaaatacaaggggccagggaataaaggagggagttatctaaaactagaagcatactagtgctaggaaatcccccatgatccctggtacacctctgcacactatgtcactattagcccaaaagaatattaacgagaatgtccacattcacaagaatttgaggccttttcccttacatcatgtccctttcttagtcacataggtaccagcaagccctatgttctagcaacattccttaactctctcatcattagttcatcaaccatgctgaccaaaaatgctccttaaagatacgaacttcacatttcccaaatatctcctgggagacctcttggcaagaaatcagcttgtttcccaactttgagaggtcatcatgaatgagaagctggagaggtcttggcacactgaccagccaaaacctttaccttaatgtgaccatcaggggatttactgggaaaattttcctatgcccttccttcatttctccctacttcctagggttgggtcaccaattactggagcatcttcagtaccggcaccttctggagcagggggaggaagaaggaatgtacagtttgctacttcttgtctatgatgggcttctcaggcactgccttgggtgcaggaggctgaaataggaggggggctgtcttctccttggcttccctggatcccattgttggaggcaccttcccagccacagttcctaggccaaacagcactggtggggccaggcttggagtggtagtggaggtggagctggaattccagggcttcatgggcaggccatttgacaggaatgccacatactggttctagaaagataggggacccatacccaccagctgagcagaaaggtcaccccagaggagtggcactgggccctccagagacagctgccagccctttttggctaggctgcaatgccaaatgtaggtgctcaggtgcacctaccaaagggaaagggagaggagagaggagggggaagaagggtcacaccagggaagctggagagggttccccttgagaaagctgcagagaatctatgttcctcaggtacaaagaatgaggagggaagaaaaattccttagggggccatccccttgtaagcacagtaatttccaagctcagggactacagaaaagccactagggacataacatgttaagaacttagagaaaaagacaaaatcagggctcataactctgggaggtccttttgtgaagctgtttctgctctgtgggacaaagagcagcaggtacagaaaaacaggctcatgggatcgtggggtcatcttttcggggaaagggggagagccctgtggaggtgatggaaggcgaacagccagggactagagaaagagcagcaatattctgagggccatggggggcaaagggctgtacctggtggtgtgccaggagcatatgcttcttgagggtagcccgctcaagaaagccctgcctgcagaaaacacaagtgaagagcggcccctccttggggtgggtcttctgatgctcctccagagctgcctgggagggaaaggcctggccacacacttcgcaggcctttctgctgctgctctctcctggctcctttctcattgcctcctgcaggctcagctcctctaccaaggtcacgctggtggcttccccttctggggtgagtgctgatgccggacttgagcttctctccggtttgcccccctcttccttgcctcctaaaacaccactgcttccctgctccattggctgaggctgatccaggctgtcaggtggtggtggtggtggcaaagaagactgttgagtagttttctcattactgtccatctccttcccagctgtggctgctgccgccactgtccccacctcctcctctgccccagatgcctcttctgaatcacctctcactgatattgccttctcacctccactctctgagcctctccctgccagggaatcttcatcagtcacatcttcctcttcttcctcatcctcctcttcctcctcctcagacaactcctcttccggtgatggctgctgggactgctgctgggggaaactccctgccccggagactgtagattgctcggagccattctcctgagcagctcctccaccttcagggagtgcagtaccaccgttggggatctggccccccaggtgcatccggacatgctgctgcagagtgacagcattggtgaacttcttctggcagatggggcaggaattctgtgcccgggcagctggactggccttgtggcccacgaaatgtgcacgcagattacccctggtggagaaggctctgccacacactttgcatttgaagggcctctcacctccatgttggccataatgaaggcgtagggcccgaggacagctaagcactcggagacagatgacacactggttaggtccagaagaggctgaggatgaaggtgcaggggcagaggtggtgggggctcctgaggcagctgaggtcaccgccacagctccttgccggtcaatcttttctaccagttgctgcagctttgatgtctcagagggtgaggcccccaagggctctagcacataggggaaggggaagctgccagtggacttgaagtggttggtaagcagtgcccagcttggtagtgaagtcaccaacttacttagttgcatgcgagttgccgtgctactttctgccactccactgatggctgagccctcactccctgggggggtgttttcatcagctttattcttgggttccactgctttcatgagcacaaacttattgaaagcagggagtcctggagccgtggctgtgcctgcactggtggagagcagagtcaggctctctgtggcactgagtgctgttgtggaggccaccagaggcttgcgctcaacccctccacctggagtggctgcctcctcctcggccttctctggtggcacggacataccataaggcaagccactgctggtaatgacatagtctaggtgctctggtactgggtgtgggttcatctgcacatgtgggtacttctcacgatgccggtggaaatgcactttgaggttgccacgggtggtaaaacggtttccacagacattgcacttatagggcctctcacccgtgtgggaacgaaggtggatctgcagggcactgtcactgccaaatactttggcacagaagcggcatttgtgccttccaccaggcttctccaagggacccatcacttctccgtagctcagctcaccacttccattctttggcttcaggagccctggggaggcagtggcctcaaggcctcgggctgccccaagacactgtgctgccagtagtcccgtggtgcttgggaatgccagatgaggcgaggcaatcagctgatctgtgctgcctggcaaggctggggaaggggcaggggtgggtttgtggcttcgcccaacccctccagcagagaaaggatgctgtgaccccagtgggtggtaaaggtggaagaaggcctgcttgggcgtttctgcccctgaagaggaagaggaggaggaggaggaagatgccagtgtcttgctggtttggacaggcttgatggggctgaagaggggtagtaggggcttggtggaagaggcagtccctgtcccaggtagctctgagggactggcaggggcacccaccgtctggcctaaggagccaagcaacagcacctgcctgcagatttgctcagtcatctgcatctgatggatctgccgctgctgcagcacccgtagctcttccaagatcagggggatattcaagtggccactgcctacccctgggggcggagggggtggtggaggaggagggggtgcaggggtcgattctggaggtaatggggttgctcccagcttgggactggccaagatcaggcccccgcctcccccagccgctgtacctgtggcagcgaccaggaaatgccctggagactcctctcctctcctctctgggccccaggtgggatccgtgggcacggaggacccagaatctggggggttgctatgctctgtgtccatgacctgaggattattgtgaccctcaggccggggttcagaggaggccgaagagttgttggggttctcctggcccccaattatcaccattacaggagggtcagtagaacatgcgttctggtgggcgaggaattcagttgggtcagtgaattgtgcgcagcacttggcacagacttgggggtgatcctcctcgctagcatcacctggggagaagacaaggagagagagcgtgggtggcgcagttgggttgggtataccgaggctctaattaacaaggaggccagtaaccgctagttgggggtggggagatgagctcaccatcagggccatgcagaagtctagagctcaggcctgatccgtgtggacaggagacaacccggcatggggcaggggggtggggagggaggaggggaggggggcaagagcatgctactcccctcctcagccaccctcccttccccaggccacaagcgagttcacggaataggtgtggggacaggggcctacgcagagaatcatgcattttctcccacccaccgaaagtcttcgccgcccctgcgcatccccctccgcccccacccctgcccagcccgaccgaccctaccgcacctccgagctctgccggctccccgcagggcaccccgagacgagagctcctctcggattcgtgcgccatggttgtgggggaagtggagggccaggtggggtgggagacaatggatattgggattgagggaggcgatggccgctgggtctgcggcagcctctgcacccagcggcccagactgcggagatggagatcggcagcggcgggggcagggagcagcggcggagggggaggggagcgaggaggcggggagaagctggagtgagaaagcggggagaggggagatctgggaggagctgatgaggaggggagtttatggggaggagctgctggggagggaggcgggagctagaggaggcgggagaagggagcgctagcgggggcgtgggggcgggagctcagagctcgggagagtttccggaggcgcagtgacaggtgctgtgaagcactgcgggggtccacctttcccggtccctggccagctccccccatctgcagatgcctttgcccaggcctaccctcctccccccgccctcccctcctaagctctaggggcacagtgggaaacgtagccctgctcagtggagcaaggcgataggcttctcttatttttctttggataaaggatccgctgagcttggaaaaagtggattccagagagggtcgtctgatctcctcagaggtctgagggccagaagaagagggggagatcagaacatccactcctcaccagcacacacaccccaaaatattcgaagttttgtctcgtctttctcacttccattcccaccctacccccatccctctccacaaaagaagtttctcagggtgggcggctgcaaggtagaatttcccaggaagtcatttcaggactctctgcggaacactaagccccttcactccccgcccctcctccccctgaataatagctgaatgcaggttactccgcagatcgcccagcctacacaacacctaattcatagagtccatgcttatttaataagccatctcctatttagtaccctcttcctcctctattctcctcttgcaacattcctcacaccgtcactattaaagacagtgggtttggggagacgctagcctgcagaggcctacggaggcccacccagctctaacctgggggggaggggagccctcttgaaacaatgcggtaggaactaccaggcagccctcagtgtctaaagccctttcagccccagcctgatttgaatgcttagaaatagctaacacctgctcaccatcacagaggcagcctcctattcagacaggataagtaagaataaaatgcctcctggaccaggtattctggcattctcttttttaccttgaaatgagtcttaaagtgcttcccacttcctaaaatactttctcttacatgcaggaagtgaccacaagtccttggttttgtggtttccctgggcatcagtaaacctaaattgttttaatcccagttctattcttgcctcactgataaaactgagacatggtggtcagtcacaccatgttataccaccgtttccctcttcataaagtggtaatattgtagctgcagtattttactcagaaaaatattgtggggacaaaaaattgaaaaattggacaatattaatgtgtaaaccaggtatggtggtgcacacttgtagccccagctacttgggaggctgaggcaggagaattgctttagtccaggagtttgaggctgcagtgagctgtgatcacacctgtgaataaccactgccctccagcttcggcaacatagtgaggccccattactttaaaaaaaaaaaaaagccgggcgcggtggctcactgtaatcccagcactttgggaggtgggcagatcacgaggtcagaagttccagaccagcatggccaacatgttgaaaccccgtctctactaaaaatacaaaaattagctgggcatgatggtacacctttaatcccagctactggggcagctgagccaggagaatggcttgaacccaggaggtggaggttgcagggggctgagatcgtggcattgcactccagcctgggcaacaagagtgaaactgcgtctcaaaaaaaaaaaaaagtctaaaaaaattaatatgtacatgtgagatttttaaagtttggggagtcctgaatttaatcaatgagataatttacattgtcagtagcaaaataatcgaagtaaccttaaatacacatatactaaaattagatctgttttccatgttgtttgttaatcttattaatttctgaggtaagatattggctaatatcagcagcatatttcaaaggtaggaagtcttttattgcagtgggtgggggagctgaaacaacctatttaaaatattagtaacatccactttacttctcaacataaattttgcctgtgtttttaaacttaaaacagtttactgaattatgttttgaaacttcagataataaggctcttagcattgtgagtcataattctgaaatggacgggttctgtgcttccaggcctggacttacaaatgagggaggggggttctatttcagtttatggcaagtcacagttttgtgcaatgtggtttatttttacagataaggaaactgaagcttggagaggttaagtgacttttccaagttcacacagtaattcagtgaagcaagcattcagaattttgactcctgtccaatgctttctcaagcacatcaactttgtatggcttccctaatgctagagaaagggccctgtgtggcttctacctgccatttgctccctggccttagtcagggagagggaatcagatggaggctttctactgagcatttgttaattagcattgaacatttgatatccagttgctgttttgtcaagtcttctgacaagaaaagaaatccttttcttttcatcttctcctgggaaacactgtcccctttcttgctctttaatgaaatgtgctttctgatgcgtaatttgatctaagctcttctttaaggtaaatttagtccctggtgaaaggtgactggatcaacagccacctgtaagaggaaccctccatttctcagtactttgcactcactgcacatcctgaaaaggggggcaggattcttacacaaacatgaatgaagtcacaaatgcaggaataaactaaactggtaatggtgtccctagatagcagataaggtgaggtaagctatctccggtcaaatgcaaagtccggggtgggactaagacctggacaagcttgtttaaacttatagagagctgaaatgacaaagaaaagggaaaccaggtggcttcccttctaaatctagtgtcccatcagattgcttctttaggcttcagagagaactgttcgggagaacaaagagaaaaataggtgagttgtatgtagcagggtgatacatttgaacagcggttttcaaattttgctgcccattaggattaccagaagagagttttaaaatttttatgtttaggtgcagtggtctgttccttgtagtcccagctactctggaggctgaggcgggaggatcacttgagctcaagggtttgagactccatctcaaaatctcaaaaaaaagaagaaaaaaaaaagaaaagaaagtttaagcacagtgggtacctcatgcctataatcctagcacttttggaggccaaggcaggaggattgcttgaggccaggaatttgagaccagcctgcataacatagtgagacccccatctctgcaaaagcaaccaaccaaccaaacttaaaaaaaatccctgtgtccaggccacatcccaggctaattaattcataatccctgaggataggatccaggcattagtttgataaagctcctcaggtgattccaatgagaatacaaagatggtgacacaatgatgagacccacatggaggactgccctttccatcataccttccaccctgctcctcacagatcttacctgagctaaacttggccacaattgggacacagacaaaatgaactctcaatgctaaatcttcccatcaggtcccctccctacagtgcccacaaccacacattaacttccttgtatcctttcccagtgaaaaatctgcttccatgaatagaatttgatataatttacaccttactgtaagtttaagtgattgcatttctttcccaggtatgggtatcttgaagcatatttttttcttttttaattgatatttgagccatatttctttttttttctttctttttttttttttttttttttttgagacggagttttgctctcattgcccaggctggagtgcaatggcatgatctcggctcaccgcaacctccacctcccaggttcaagcgattctcctgcctcagccttcccaagtagctgggattacaggcatgtgccaccaagcccggctaattttgtatttttagtagagatggggtttctccatgtgggtcaggctggtctcaaactcccgacctcaggtgatctgccaacctcggcctcccaaagtgttgggattacaggcgtgagccatcgcgcccggccaccatatttctaattgtaaggtgaaaggctttgttctacagagttcaagcatcatccacccattaaggctggagtgaagtggcacaatcatagctcactgcagactctacctcccaggcttaggtgatcctcccacctcagcttcctgaatagctgggactacaggcatgcacaatcatgcccagctaattaaaatatttttttctgtagagatgaggtttcactatgttgcccaggctgtctggaatacctgggctcaagggatcctcctgccttgtccccacaaagtgctgagagtacagatgtaagccactgcctctggcccacttacttattattgacactgaacaatgctaattggtagcttccataattatgaattgattctgtaactattgctactgactacttcttagggaaatatctcatcttctcctccttactcctctttcctaaatgtagacacataataatcctttgcaacccagacctactaatgtaactatggcctatgtaacacagtagactaacaggcacaatgattggtacacctggtgctaagtgagaaaaagatatttgtttccagaacaggaatatcttagatcaaacataagaatgttcttttaatgaaaatttctttgacttcaaaggactcaacacttaacatggaattcataccattttggagctgggacttcagagatctgacactctcattgtcattgtgcacagtgattcagacctgagttaaagtcccagctctagaacattctaatatttgtgatcttgggaaaatttcttaatctctcccagagtttgttttcttatttttttttgggacagagtttcactcttgttgcccaggctggagtgcaatggcacgatcttagctcaccgcaacctccgcctcccaggttcaagcgattctcctgcctcaccctccctagtagctgggattacaggcatgtgccaccacgcccggctaattttgtatttttttagtagagacggggtttcttcatattgctcaggctggtctcaaactcccagcctcaggtgatctgcccacctcggcctcccaaagtgctggattacaggcatgagccaccgcgcctggccagccttttttttttttttgagacggagtctcgctctgtcgcccaggctggagtgcaatggtcgcccaggctggagtgcaatggtgtgatctcggctcactgcaatctccgcctcctgggttcaaacgattttcctgcctcagcctcccaagtagctgggattacaggtgtgcgccatcacacccagctaatttttgtatttttagtagagatgaggtttcaccttgttggccaggctggtcttgaactcctgacctcaagtgatttgcccacctcagcctcccaaagtgctgagattacaggcatgagctgctgtgcccggctgatttctcttctttaaaatgagggtactgccatacaaaggaaggaaattctgatacatgctacaacatgaatgaactttgtaaacattatgctttcagacaaatttgactttaattgagaaaaaaagagaaaacatactaagtgcaataaagcagacacaaaaggacaaatattgtatgattccattagtatgaggtacccaaacattatatgagtccattaatatgaaatttggcaaggtcacacatacagaaagcagagtagaggctaacagggctaagggaatgggagaatggggatttattgtttaacggttacagtttctgtttgatgatgaaaaagatattgaaacagcagtaatggttacataacatagtgaatgtacttaatgccactgaattgtacacttaaaaatggttaaaatggtaaattttattacacatattttacaataaaaaaattttagccaggtgtggtggcatgcacctgtaatcccagctgttcaggaggctgaggcaggagaatctcttgaaccctggaggtggaggtttcagtgagccgagacgtgccactgcactccagcctgggcaacagagtaggacttggtctcaaaaaaagaaaaaaattttttttgtaataataagggagttggggctgggcgtggtggctcacgcctgtaatcccagcactttgggaggccaaagtgggcggatcatgaagtcaggagatcgagaccatcctggctaacacagtgaaaccctgtctctactgaaaatataaaaaattagccaggtgtggtggcgggcgcctgtagtcccagctacttgggaggctgaggcaggagaatggtgtgaacccgggaggcggagctcgcagtgagccaagatcgcgccactgcaccccagcctgggcgacagagcgagactccgtctcaaaataataaaaataaataaataaataaataaaataataataataacggagttgggaggaaaaagaggaaatgcaaaaagggcctagcacagtacctgaatgctccacaaatattagccatgggtgttagttattatttgaatgtcaaaagctgaatgaagccctggggtaagaaaggtcacatgtgcccaaggtcacatagcttcaaggtccacactagattgaaaaccaagttttctgttttcttatctagtactctgtaacaccaggactgagatactctctattccaaaatgtgttttttctgatctgggaatacctaggttgagtggcccagggatcaataacctgagagatgaggctctttacttccaaatgtaaacagagcccccaaaactctacctttgccttctttcctctcttgctgttcttgctatctgccaacttccatctaaagtactcccctctctccctctagatctgtttggctgctgtcctggtttcttcttctcactaaatatctgggtttctgattgtttcctttatttcccagatgtactggtttgcatttttcccccagtcacatcctttgtgttctctaatccagatttctagactctgtaggggagagagaaggtttttttttttcctctctagagtttttaagtgaatagagtatttcctgcccatcacttatatgcaataactgttctgttaggttttgatgctctggttagggaagctgagcaaaaacggctggaaaacagatttttcagactgtttcttggtgatgtcttaggtcactgcagaattttggcttttaaaatatgtaacaaaggctcagcatttgcatgttgtatatggcacatattgcttacaagaaggcaaaagactcctggaaacattactggcaccctagactactgactaaatgtcttctgatactcatgatgatatccataatttcacaggtacaccaaaggatacatgtgcccctaaataagagcccttcctccctaactgtggagcatgctctggggtagaaggaagtcagatgcctgaagatcacataagtgaatagaaaccctgtctataaaaaattagggaaaaggagagctctcattctgttttgcagaatggatgctgccccattcatgattaagaaaatttattaatttaaaagaaaaccagaaaatgtgaaatttatatattataagcttataagatccaggaggaattttagatacgatcaaatagagccacctcattttgcagatgaggcccaatgacatccagatcataagtagcctaggatctttcactccaggggaattctgatgagaaaatccttaggctttcttacggtagatcttaacagagggtgctactgcttccttgctccttacatttgttcctgcctttcatagctcaaaggcaaattttcatcaaaaatttgttgatgccattgggtttaaacctttactgtttctatggggatggctttgtaacagcattaccatgcccccaggtggaagctatatcttaaagggcttgaaaatccattcaagacagccgctaaagatagcttttgactccctcacagaagatttttcctcagctatgatatggggaatgggtgagcagatgggagaagtaggaagaagaggagagaatgcttcttgggggtttggaggggtgttcagcatagttccacaatcaaaccagcaggagagcagaactgtgaggcaactctggggaggagttgaggctctaggggaagtctcctgtagagcacaagcaggaaacatccggcctatagcagcattaagaagggctaatgtgtctcaggagggaaggatgccatcaccatagaacctctaaatatgggcacagtaggatcccagaaaagcagtgtttcggggaggatgcgttctgcccaaaacatgtctgttaaggttattttgtagcacatggagcgctgatttgacctcaagtttttgttttttaacaggtggaaaggcaagtttaatctacaattttagtcgccaccaatacactctcttagagcttttcatgacacgtctcataaagaaatgctgatggccgggagcggtggctcacgcctgtaatcccagcactttgggaggccaaggcgggcagattacgagatcaggagatccagagcatcctggctaacacggtgaaaccccgtctctactaaaaatacaaaaaattagcccggcgtggtggcaggcgcctatagttccagctactcgggaggctgaggcaggagaatggcgtgaacctgggaggtggagcgggcagtgagccaagattgcaccactgcactccaacctgggcgacagagcgagactctctctcaaaaaaaaaaaaagaaaaaaagaaaaagaaaagaaaagaaaaaaaaagaaatgctgacgtttgccaagaggttcctgagttttggtcatactacagcacttgcaggcagtgtcactgcattcacatataatgataataacgatattcacacatattaagcacttatttatgctaggtatttttccaagggatttacacatattaactcatttagattttcacaacaacctaatgaggtagctagtatacacatctttatttcacagatgaggaaactgaagcatagagaggcaaaataaaccagccaaggtcacatagctaaccaagtggtggagctgggatttgtctaaaagtctggtttcagaacccttgtgcttaatcctatactatactgttgggtgtatcaactgtatgctaaacagttgcctgtctggagccaggacttccagactttcagtctgcacatatggagccataccactgacaagtatgtccaaaacttctttgatcctaagaattacctggacaattgcaaaatatatagattcccacaccctggctcagatgtactcacaatcaggcaagtttttaaaacccaggtttagtgggtttagtgagcactaccagccagccctgagcattaggaaattgaagtttttgtcctgattttgcttctgtctctcagactctgagcaatttcactcttcaattccctgcttgctctactgtctgcctgtcacttaacggaatgttacaagaatacatacaatttttccccctcataagggacacctgttgcttcaaaaacacggtatcctcataaaatgatatgcatgtagtaacaggtgtattttcttgcacttcttttgttttgttttgttttttgctttgctttccttgaagcacaaacctaagcccctcatccagacctagccttcagctgtcctccaggtgacacgcatacacaccccaaaccaggctgcattctgaccgaccttagctctctccctctgggagctctgatcggctctcagttcagcccaacaatgagaaacttttttctcgtctccctcaggggagccttcacgtttatccaattcattctcttgcaacccaactctccagaaagaaaaggggggaaaatcccaccccgaagagacggtcttcaggtctgaggacgttacttagcaacggcacaaagaccagtgagcaaagggagacctgaggagaaaactcttgggtggggagacagagccagtttgaaaactccatttcatccagagaaaaacaaggaaaacacaaacagaatcaatcccaagtaacaagcggggcttctccccagcgcaggtcatctcttactccctgcatctcaactccttcaaacccccagtgaccaagtccgcccccgcctggtttcgcccatggcccgagtgccctccccttgccctggcctgacccacacaggcttggacttaggggcccccacccctccccaggcacccaccgttctcagacgcgctgggaccttcgcagtccgagattaactgttggggtttccgctgctttcgccgagacattcccgggtagagagttgggagagggaggggcaacgctcacttggtcttaaccggggtgacctggtctcgtctcccccttgggtccgaagccaattgatgcctctcccccagcgcaaatcactgtgaagcagagatgttcttctttcccagagacacagactctctctctctctctgattctctgttcttgactctctctct

SEQ ID NO: 3 - protein, 1002 aa, Mus musculus
MAQETGSSSRLGGPCGEPAERGGDASEEHHPQVCAKCCAQFSDPTEFLAHQNSCCTDPPVMVIIGGQENPSNSSASSAPRPEGHSRSQVMDTEHSNPPDSGSSGPPDPTWGPERRGEESSGQFLVAATGTAAGGGGGLILASPKLGATPLPPESTPAPPPPPPPPPPPGVGSGHLNIPLILEELRVLQQRQIHQMQMTEQICRQVLLLGSLGQTVGAPASPSELPGTGAASSTKPLLPLFSPIKPAQTGKTTASSSSSSSSSGAEPPKQAFFHLYHPLGSQHPFSVGGVGRSHKPTPAPSPALPGSTDQLIASPHLAFPGTTGLLAAQCLGAARGLEAAASPGLLKPKNGSGELGYGEVISSLEKPGGRHKCRFCAKVFGSDSALQIHLRSHTGERPYKCNVCGNRFTTRGNLKVHFHRHREKYPHVQMNPHPVPEHLDYVITSSGLPYGMSVPPEKAEEEAGTPGGGVERKPLVASTTALSATESLTLLSTGTSTAVAPGLPTFNKFVLMKAVEPKSKADENTPPGSEGSAIAGVADSGSATRMQLSKLVTSLPSWALLTNHLKSTGSFPFPYVLEPLGASPSETSKLQQLVEKIDRQGAVAVASTASGAPTTSAPAPSSSASGPNQCVICLRVLSCPRALRLHYGQHGGERPFKCKVCGRAFSTRGNLRAHFVGHKTSPAARAQNSCPICQKKFTNAVTLQQHVRMHLGGQIPNGGSALSEGGGAAQENSSEQSTASGPGSFPQPQSQQPSPEEEMSEEEEEDEEEEEDVTDEDSLAGRGSESGGEKAISVRGDSEEVSGAEEEVATSVAAPTTVKEMDSNEKAPQHTLPPPPPPPDNLDHPQPMEQGTSDVSGAMEEEAKLEGISSPMAALTQEGEGTSTPLVEELNLPEAMKKDPGESSGRKACEVCGQSFPTQTALEEHQKTHPKDGPLFTCVFCRQGFLDRATLKKHMLLAHHQVPPFAPHGPQNIATLSLVPGCSSSIPSPGLSPFPRKDDPTMP

SEQ ID NO: 4 - DNA, 4547 bp, Mus musculus
atggcgcaggaaaccgggagcagctctcgactcgggggaccctgcggggagcctgcggagcgcggaggtgatgctagcgaggaacaccacccccaagtctgtgccaaatgctgcgcacaattttctgacccgaccgaattcctcgctcaccagaactcatgttgcactgacccaccggtaatggtgataattggaggccaggagaatcccagcaactcttcagcctcctctgcgccccgaccagagggccacagtaggtcccaggtcatggatacagagcacagcaatcccccagattctgggtcctctgggcccccggatcccacttgggggccagagcggaggggagaggaatcttctgggcaattcctggtcgctgccacaggtacagcggctgggggaggtgggggccttatcttggccagtcccaagctgggagcaaccccattacctccagaatccactcctgcaccccctcctcccccaccaccccctccccctccaggtgtaggcagtggccacttgaacattcctctgatcttggaagagttgcgggtgctgcagcagcgccagattcaccagatgcagatgactgaacaaatctgccgccaggtgctgctacttggctccttggggcagaccgtgggtgcccctgccagtccctcagagctacctgggacaggggctgcctcttccaccaagcccctcctgcctctcttcagtcccatcaagccagcgcaaactggcaagacactggcatcttcctcttcgtcatcctcctcctctggagctgaaccgcctaagcaggctttcttccacctttaccatccactgggatcacagcatcctttctctgtaggaggggttgggcggagccacaaacccacccctgccccttcccctgcgctgccaggcagtacggatcagctgattgcttcacctcatctggcattcccaggcaccactggactcctggcagctcagtgtcttggggcagcaaggggccttgaggctgctgcctccccagggctcctgaagccaaagaacggaagtggtgaactgggctatggggaagtgatcagttccttggagaaacccggtggaaggcacaaatgccgcttttgtgcaaaagtattcggcagtgacagcgccctgcagatccaccttcgttcccacactggtgagaggccctataagtgcaacgtctgtggtaaccgtttcacaactcggggcaacctcaaagtacattttcaccggcatcgtgagaagtacccacatgtgcaaatgaatccacatccagtaccggagcacctagactacgtcatcaccagcagtgggctgccttacggaatgtctgtgccaccagagaaagcagaagaggaggcaggcacaccaggcggaggtgttgaacgcaaacccctagtggcctccaccacagcactcagtgccacagagagcctgacactgctctccactggcacaagcacagcagtggctcctgggctccctactttcaacaagtttgtgctcatgaaggcagtggaacccaagagtaaagcggatgagaacacgcccccagggagtgagggctccgccatcgctggagtagcagacagtggctcagcaacccgaatgcagctaagtaagctggtgacgtcactaccgagttgggcactgcttactaatcacttgaagtcaactggaagtttccccttcccttatgtgctagaacccttgggggcttcgccttctgagacctcaaagctgcagcagctagtagaaaagattgaccgccaaggagctgtggcggtggcatctactgcctcgggagctcccaccacttctgcccctgcaccttcctcctccgcttctggacctaaccagtgtgtgatctgtcttcgggtcctgagctgccctcgggctctacgcctgcattatggccaacatggaggtgagcggcccttcaagtgtaaagtgtgtggccgagctttctccacaaggggcaatttgcgcgcacatttcgtgggtcacaagaccagtccagctgcccgggctcagaactcctgccccatttgtcagaagaagttcactaatgctgtcactctgcagcaacatgttcggatgcacctggggggccagatccccaatgggggttccgcactttctgaaggtgggggagctgcccaggaaaacagctctgagcagtctacagcctctggaccagggagtttcccccagccgcagtcccagcagccatctccagaagaggagatgtctgaggaagaggaagaggatgaggaagaggaggaagacgtgacagatgaagattccctagcaggaagaggctctgagagtgggggagagaaggccatatcagtacgaggtgactcagaagaggtatctggggcagaggaagaagtggcaacatcagtagcagcacccaccactgtgaaggagatggacagtaatgagaaagcccctcaacacactctgccgccacctccgccaccacccgacaacctggatcatccccaacccatggagcagggaaccagtgatgtttccggagccatggaggaagaagccaaactggagggaatctcaagcccgatggcagccctcacccaagaaggggagggcaccagcacccctttggtggaagagctgaacttaccggaagccatgaagaaggatccaggagagagcagcggcaggaaggcctgtgaagtatgtggccagagctttcctacccagacagctctggaggagcatcagaagacccatcccaaggatgggccactcttcacttgtgtcttctgcaggcagggcttccttgaccgtgctaccctcaagaagcacatgctgttggctcaccaccaggtaccgccctttgcaccccatggccctcagaatattgctactctttccttggtccctggctgttcctcctccatcccttctccagggctctccccattccctcgaaaagatgaccccaccatgccatgagcctgctttctgtacctggtcctctatgacccagagagcagaaacctgagagcttcatagaggaactccaagatttactcaccctcctcttgtcctttctcaagtcctgacatgatgtttctagtggcttcttctctagtccctgagcttgacaattgcctttgaaagagaatgtccccttaagaaatttttatcacctttttgttctgtgtaactaagggaaacaaattccctatagcttttacattctcaagggggagctctctcctcttctccctttccctttggcaggtatactagaacccccatccttggagtggcagccttggtccaaggggctggcaactgtccatggaaggcccagcgttactccttggtgatcttgaccaccctgcaagactttctagggccgggaccttcttgagaagcttgtaaggggtggtaggtttctttctgcaaccactacccagttttccactgagccctggagttctggacctacctgcattgccactcgggccctagtaccatcattgctgtgaaagcccaggaactgtgtttcacaaggtgactccagtgacatgatccagagaggcaaagaacatagcctccggaagttgaggctgtgcccaacaagcacaccggaagaaagaagaaactataacttctttctccttcccccctgctccagagagtgctggcaataaagatattctagcaattggtgactcaccctagaaggtagggacaagtgaaggactgggaccctttttgcagtatgttccttgactcgccacattgaggcaaagatagtggctggtcaagatgccaggactactccagcttcccatcatgtcctctcaaccaacaagcaggtttcctaccaagaggtctctcgtgtgatagtttagggagtatgaagtttctaactctaaagaatcctgttggtgaggatgattatttaagcaatgatggggagttgagggttgttgctaaaacaggcattgctgggaatctatttgatgaagaacaggacttgatgtaaggggactcgatgttcagctcttgtgagtatgaacgttttctttgagctaatggtgatgtggtatgcagaggtaccaggggccatgggggtgtgtgtgcttcctgtcactagaatgtttttagttttagatgactccctattttattccctcaccccttgtatttcccttgctgtcttctcaaaacccctttcctcccccagttttgcctgaccatgggccagagcttatgtcttattttttttctagaagttgagagacagagcttcaagtggtttccccccgtctctgtcttgtagtgagatgtagtatttactcttaacataggatcctgtggaacaggtgttctgagaagactgaattttgctgttagctgttgtcaatgatgattctctaaagtagtgggctccagagctccctaacacagtgaaatgtgtaagagccgagaggggagatactagaattttttccttcatcattaaaggtgttttggct

SEQ ID NO: 5 - DNA, 22 bp, Artificial Sequence
  derived from human Sal2 gene
ccacaaccatggcgaatccgag

SEQ ID NO: 6 - DNA, 24 bp, Artificial Sequence
  derived from human Sal2 gene
ggtgatggaaggcgaacagccagg

SEQ ID NO: 7 - DNA, 26 bp, Artificial Sequence
  derived from human Sal2 gene
cttgttaattagagcctcggtatacc

SEQ ID NO: 8 - DNA, 22 bp, Artificial Sequence
  derived from human Sal2 gene
gcacggaggacccagaatctgg

SEQ ID NO: 9 - DNA, 63 bp, Polyoma virus
gatatactttgtaatgtgcaagaaggcgacgaccccttgaaggacatatgtgaatatagctga

SEQ ID NO: 10 - protein, 20 aa, Polyoma virus
DILCNVQEGDDPLKDICEYS

SEQ ID NO: 11 - protein, 19 aa, TMD25 mutant Polyoma virus
DILCNVQEDFVMCKKATTP

SEQ ID NO: 12 - DNA, 60 bp, TMD25 mutant Polyoma virus
gatatactttgtaatgtgcaagaagactttgtaatgtgcaagaaggcgacgaccccttga

SEQ ID NO: 13 - protein, 16 aa, Polyoma virus
NVQEGDDPLKDICEYS

SEQ ID NO: 14 - protein, 14 aa, Artificial Sequence
  dervived from Polyoma virus large T antigen
NVQEGDDPLKDICE

SEQ ID NO: 15 - protein, 10 aa, Artificial Sequence
  dervived from Polyoma virus large T antigen
NVQEGDDPLK

SEQ ID NO: 16 - protein, 7 aa, Artificial Sequence
  dervived from Polyoma virus large T antigen
NVQEGDD

SEQ ID NO: 17 - protein, 4 aa, Artificial Sequence
  dervived from Polyoma virus large T antigen
NVQE

SEQ ID NO: 18 - protein, 15 aa, Artificial Sequence
  dervived from Polyoma virus large T antigen
NVQEGDDLKDICEYS

SEQ ID NO: 19 - protein, 15 aa, Artificial Sequence
  dervived from Polyoma virus large T antigen
NVQEGDDPKDICEYS

SEQ ID NO: 20 - protein, 15 aa, Artificial Sequence
  dervived from Polyoma virus large T antigen
NVQEGDDPLDICEYS

SEQ ID NO: 21 - protein, 13 aa, Artificial Sequence
  dervived from Polyoma virus large T antigen
NVQEGDDDICEYS